(19) United States
(12) Patent Application Publication
(10) Pub. No.: US 2007/0299319 A1
(43) Pub. Date: Dec. 27, 2007

(54) Cognitive Training Using A Continuous Performance Adaptive Procedure

(75) Inventors: Samuel C. Chan, Alameda, CA (US); Joseph L. Hardy, Richmond, CA (US)

Correspondence Address: HUFFMAN LAW GROUP, P.C., 1900 MESA AVE., COLORADO SPRINGS, CO, 80906, US

(73) Assignee: Posit Science Corporation, San Francisco, CA (US)

(21) Appl. No.: 11/760,413

(22) Filed: Jun. 8, 2007

Related U.S. Application Data
(60) Provisional application No. 60/804,427, filed on Jun. 9, 2006.
(60) Provisional application No. 60/868,839, filed on Dec. 6, 2006.

(51) Int. Cl. A61B 5/00

(52) U.S. Cl. 600/300

(57) ABSTRACT

A computer-implemented method for enhancing cognition, e.g., for improving cognitive ability and memory of a subject using continuous performance with respect to a stimulus stream. A target descriptor is presented to the subject after which, a continuous sequence of stimulus groups from a stimulus set is presented one stimulus at a time, each stimulus presented for a specified duration, and separated by a specified inter-stimulus-interval (ISI). For each stimulus in the sequence: the subject is required to respond to the stimulus, indicating when the stimulus corresponds to the target descriptor, and the duration and/or the ISI adjusted accordingly, using an adaptive procedure. The presenting the target descriptor(s), presenting the continuous sequence, and requiring/determining/adjusting for each stimulus, are repeated iteratively to improve the cognition of the subject.

### CROSS REFERENCE TO RELATED APPLICATION(S)

This application claims the benefit of the following US Provisional Patent Applications, which are incorporated herein in their entirety for all purposes:

| Docket No. | Serial No. | Filing Date: | Title: |
|---|---|---|---|
| PS.0128 | 60/804,427 | Jun. 9, 2006 | Aristotle 1-3 |
| PS.0131 | 60/868,839 | Dec. 6, 2006 | COMPUTER BASED |
| | | | TRAINING PROGRAM |
| | | | TO IMPROVE SUS- |
| | | | TAINED ATTENTION, |
| | | | INHIBITION, WORKING |
| | | | MEMORY |

The following applications are related to the present application, and are hereby incorporated by reference in their entirety for all purposes:

| | | | |
|---|---|---|---|
| PS.0231 | *** | *** | COGNITIVE TRAINING USING |
| | | | ONE OR MORE STIMULUS |
| | | | STREAMS AND TARGET |
| | | | ATTRIBUTE MATCHING |
| PS.0232 | *** | *** | COGNITIVE TRAINING USING |
| | | | MULTIPLE STIMULUS STREAMS |
| | | | WITH RESPONSE INHIBITION |

### FIELD OF THE INVENTION

This invention relates in general to the use of brain health programs utilizing brain plasticity to enhance human performance and correct neurological disorders, and more specifically, to a method for improving cognition using stimulus streams.

### BACKGROUND OF THE INVENTION

Almost every individual has a measurable deterioration of cognitive abilities as he or she ages. The experience of this decline may begin with occasional lapses in memory in one's thirties, such as increasing difficulty in remembering names and faces, and often progresses to more frequent lapses as one ages in which there is passing difficulty recalling the names of objects, or remembering a sequence of instructions to follow directions from one place to another. Typically, such decline accelerates in one's fifties and over subsequent decades, such that these lapses become noticeably more frequent. This is commonly dismissed as simply “a senior moment” or “getting older.” In reality, this decline is to be expected and is predictable. It is often clinically referred to as “age-related cognitive decline,” or “age-associated memory impairment.” While often viewed (especially against more serious illnesses) as benign, such predictable age-related cognitive decline can severely alter quality of life by making daily tasks (e.g., driving a car, remembering the names of old friends) difficult.

In many older adults, age-related cognitive decline leads to a more severe condition now known as Mild Cognitive Impairment (MCI), in which sufferers show specific sharp declines in cognitive function relative to their historical lifetime abilities while not meeting the formal clinical criteria for dementia. MCI is now recognized to be a likely prodromal condition to Alzheimer's Disease (AD) which represents the final collapse of cognitive abilities in an older adult. The development of novel therapies to prevent the onset of this devastating neurological disorder is a key goal for modern medical science.

As a specific example, it is believed that older adults suffer from a degraded ability to attend effectively to a specific task as well as to inhibit an unwanted action, i.e., a degradation of executive function and cognitive control. This deficit may manifest itself psychophysically in the subject's poor ability to identify correctly a given target in a temporal continuous stream of stimuli, and behaviorally in the subject's poor ability to recall events over short time span.

The majority of the experimental efforts directed toward developing new strategies for ameliorating the cognitive and memory impacts of aging have focused on blocking and possibly reversing the pathological processes associated with the physical deterioration of the brain. However, the positive benefits provided by available therapeutic approaches (most notably, the cholinesterase inhibitors) have been modest to date in AD, and are not approved for earlier stages of memory and cognitive loss such as age-related cognitive decline and MCI.

Cognitive training is another potentially potent therapeutic approach to the problems of age-related cognitive decline, MCI, and AD. This approach typically employs computer- or clinician-guided training to teach subjects cognitive strategies to mitigate their cognitive and memory loss. Although moderate gains in memory and cognitive abilities have been recorded with cognitive training, the general applicability of this approach has been significantly limited by two factors: 1) Lack of Generalization; and 2) Lack of enduring effect.

Lack of Generalization: Training benefits typically do not generalize beyond the trained skills to other types of cognitive tasks or to other “real-world” behavioral abilities. As a result, effecting significant changes in overall cognitive status would require exhaustive training of all relevant abilities, which is typically infeasible given time constraints on training.

Lack of Enduring Effect: Training benefits generally do not endure for significant periods of time following the end of training. As a result, cognitive training has appeared infeasible given the time available for training sessions, particularly from people who suffer only early cognitive impairments and may still be quite busy with daily activities.

As a result of overall moderate efficacy, lack of generalization, and lack of enduring effect, no cognitive training strategies are broadly applied to the problems of age-related cognitive decline, and to date they have had negligible commercial impacts. The applicants believe that a significantly innovative type of training can be developed that will surmount these challenges and lead to fundamental improvements in the treatment of age-related cognitive decline. This innovation is based on a deep understanding of the science of “brain plasticity” that has emerged from basic research in neuroscience over the past twenty years, which only now through the application of computer technology can be brought out of the laboratory and into the everyday therapeutic treatment.

Thus, improved systems and methods for improving cognition and memory are desired.

### SUMMARY

Various embodiments of a system and method for enhancing cognition and memory in a subject, specifically, executive function and cognitive control, via cognitive training exercises using continuous stimuli are presented. Embodiments of the computer-based exercises or tasks described herein may operate to renormalize and improve the ability of the nervous system to perceive, process, operate on, and remember information presented in a continuous manner. This may be achieved by having subjects perform any of various tasks using stimulus streams under conditions of high engagement/stimulation and under high reward for correct performance in order to encourage renormalization of cognition and memory.

A set (or sets) of stimuli may be provided for presentation to the subject. For example, the stimuli may be stored on a memory medium of the computing device, on a memory medium coupled to the computing device, e.g., over a network, etc. The stimuli may include various types of stimuli, e.g., visual stimuli, e.g., orthographic words or numbers, pictorial stimuli, e.g., geometric shapes with various attributes, and so forth, as well as auditory stimuli, e.g., spoken words or phrases (via human recordings or synthesized speech), sounds, etc. In some embodiments, stimuli may be provided in multiple different modes, e.g., auditory, orthographic (textual), or pictorial stimuli may be used, among others. Note that as used herein, a “more difficult stimulus” means that in the context of a cognitive training task, the presentation of the stimulus would result in a lower probability of correct response by the subject.

A target descriptor may be presented to the subject. The target descriptor may be presented visually and/or audibly as desired, and may correspond to one or more stimuli from the set of stimuli. In various embodiments, the target descriptor may specify any of various matching or correspondence criteria, e.g., target correspondence, target attributes, correspondence condition, and so forth, indicating to the subject under what conditions the presented stimuli may be considered to correspond or match, e.g., with respect to the target (e.g., in the case of target attributes), or among themselves (e.g., in the case of a target correspondence). In other words, the target descriptor may specify target attributes and/or relationships among the stimuli, or any combination. Any type of target descriptors may be used as desired. Various exemplary tasks are described below illustrating exemplary target descriptors, although it should be noted that any types of target descriptors may be used as desired, e.g., arbitrary attributes of arbitrary stimuli.

For example, in some exemplary embodiments, the target descriptor may specify one or more of: a word or object (e.g., boy, dollar), a category (e.g., clothing, animal), one or more attributes (or the absence of attributes) (e.g., color, quantity, etc., or the absence of a color, quantity, etc., e.g., “not blue”). In one embodiment of a word/object matching task, the subject is to seek out the target object from a stream of simple objects consisting of the target objects and non-target objects, and indicate matches. In one embodiment of a category matching task, the subject is to indicate when a stimulus matches or corresponds to the target category. In one embodiment of the attribute(s) task, the subject is to indicate when a stimulus matches or corresponds to the target attribute(s). Note that the target descriptor may also be compound, e.g., may include multiple objects, categories, and/or attributes, or any combination of such. Thus, a target descriptor may itself include multiple target descriptors. For example, an exemplary compound target descriptor may be “not Shrek, green monsters with two shoes”. Thus, a target descriptor may itself include multiple target descriptors.

Other exemplary target descriptors may specify attribute relationships. For example, the target descriptor may specify one or more “identical” or “distinct” attributes, among one or more stimulus attributes, e.g., color, shape, texture, quantity, and the absence or negative of any of these attributes (e.g., “not blue”). Example target attributes of this type include: “identical color”, meaning that all the stimuli in the stimulus group have the same color, and “identical shape and distinct quantity”, meaning that all the stimuli in the stimulus group have the same shape, but each stimulus in the group has a different quantity or number of elements, and so forth.

A continuous sequence of stimulus groups from the set of stimuli may be presented to the subject one stimulus at a time, where the continuous sequence of stimulus groups includes one or more stimuli corresponding to the target descriptor, and one or more stimuli not corresponding to the target descriptor. The stimuli that do correspond to or match the target descriptor may be referred to as corresponding or matching stimuli, target stimuli, or simply targets. Those that do not match or correspond to the target descriptor may be referred to as “foils”, non-matching or non-corresponding stimuli, non-target stimuli, or simply non-targets.

Each stimulus group may be presented for a specified duration, and the stimulus groups in the continuous sequence of stimulus groups may be separated by a specified inter-stimulus-interval (ISI). Note that in embodiments where each stimulus group includes a single stimulus, the continuous sequence of stimulus groups composes, includes, or corresponds to a continuous stimulus stream, i.e., each stimulus group is simply a single stimulus. Similarly, in embodiments where each stimulus group includes one or more stimuli, the continuous sequence of stimulus groups includes or corresponds to one or more continuous stimulus streams, where the one or more stimuli in each stimulus group are from respective continuous stimulus streams of the one or more continuous stimulus streams. In other words, each stimulus group includes stimuli from respective (one or more) stimulus streams, and the presenting of the sequence of stimulus groups includes presenting the one or more stimulus streams to the subject, with each stimulus stream contributing a respective stimulus for each stimulus group.

The particular stimuli presented may be dependent on the particular task being performed. For example, the stimuli for the exemplary word/object matching task may include various words or objects (e.g., presented textually or pictorially), the stimuli for the exemplary category matching task may include objects in the class of the category (e.g. for category “animal”, target stimuli may include “dog”, “ant”, etc.) and objects that do not fall into the category (e.g. “box”, “shoes”, etc.), and, following the example above, with the attribute “blue figure”, the stimuli for the exemplary attribute(s) matching task may include a set of geometrical objects (e.g. circle, triangle, square) of various colors (e.g. blue, green, red), where the subject is to respond only when figure of the desired color is shown. Note, however, that in other embodiments, stimuli in accordance with any other types, modes, or categories may be used as desired. Note further that, as mentioned above, in various embodiments, the continuous sequence of stimulus groups may be presented in a specified mode or modes, including one or more of: an auditory mode, wherein each stimulus or stimulus group is presented audibly, a pictorial mode, wherein each stimulus or stimulus group is presented pictorially, or an orthographic mode, wherein each stimulus or stimulus group is presented textually, among others.

As mentioned above, in one embodiment, the at least one stimulus in the stimulus group may include a plurality of stimuli, where the target descriptor includes or specifies one or more relationships of attributes of the plurality of stimuli. Each relationship may include one or more of: identical, where each stimulus of the plurality of stimuli has a common attribute value, or distinct, where each stimulus of the plurality of stimuli has a different attribute value. Moreover, as also indicated above, in some embodiments, the attributes of the plurality of stimuli may include two or more of: color, shape, texture, quantity, absence of a specified color, absence of a specified shape, absence of a specified texture, or absence of a specified quantity, among others. Note that in other embodiments, any attributes, combinations of attributes, absence of attributes or combinations of absences of attributes, or any combination of such, may be used as desired. Note that in some embodiments, the target descriptor(s) may be presented concurrently with the stimulus (i.e., on the same screen or with sound), while in others, the target descriptor(s) may be presented prior to the stimuli.

Each stimulus group in the continuous sequence of stimulus groups may be processed as follows:

The subject may be required to respond to the stimulus group by indicating when the stimulus group (i.e., all the stimuli in the stimulus group) corresponds to or matches the target descriptor. In some embodiments, the subject may be required to respond differently when the stimulus group does not match or correspond to the target descriptor, or to refrain from indicating when the stimulus group does not correspond to the target descriptor. Said another way, in these embodiments, when the stimulus group matches or corresponds to the target descriptor, the subject should indicate that this is so. However, when the stimulus group does not match or correspond to the target descriptor, in some embodiments, the subject should indicate so with a different response, while in other embodiments, the subject should refrain from responding. As noted above, “corresponding” or “matching” stimuli, refer to stimuli that correspond to the specified target descriptor(s). Thus, for example, in some embodiments where the subject is to respond positively when the stimulus group matches, and negatively when the stimulus group doesn't match, the subject may press a first key, e.g., an up arrow key, when the stimulus group matches or corresponds to the target descriptor, and may press a second key, e.g., a down arrow key, when the stimulus group doesn't match. Of course, other keys or GUI controls may be used as desired. In embodiments where the subject is to inhibit responses to non-target stimuli, when the stimulus group doesn't match or correspond to the target descriptor, the subject should do nothing.

Thus, in a preferred embodiment, one or more stimulus streams (continuous sequence of stimulus groups) may be presented on a visual display device, e.g., a computer monitor, where the subject may be required to respond when the stimulus group matches or corresponds to the target descriptor, e.g., by pressing the spacebar on the keyboard, clicking on a GUI control, etc.

A determination may be made as to whether the subject responded correctly for the stimulus group. For example, in some embodiments where the subject is to inhibit responses to non-targets, the subject's response to the stimulus group may include one of: a correct response, including: a true positive, where the subject correctly indicates when the stimulus group (i.e., the at least one stimulus in the stimulus group) corresponds to or matches the target descriptor, or a true negative, where the subject correctly refrains from indicating when the stimulus group does not correspond to or match the target descriptor; or an incorrect response, including: a false negative, where the subject (incorrectly) fails to indicate when the stimulus group corresponds to or matches the target descriptor, or a false positive, where the subject incorrectly indicates that the stimulus group corresponds to or matches the target descriptor. In other words, in these embodiments, if the stimulus group corresponds to or matches the target descriptor, the correct response is to indicate the correspondence or match, and the incorrect response is to fail to indicate the correspondence or match, and if the stimulus group does not correspond to or match the target descriptor, the correct response is to refrain from indicating a correspondence or match, and the incorrect response is to (incorrectly) indicate that the stimulus group corresponds to or matches the target descriptor.

In some embodiments where the subject is to respond by indicating both targets and non-targets, the subject's response to the stimulus group may include one of: a correct response, including: a true positive, where the subject correctly indicates when the stimulus group (i.e., the at least one stimulus in the stimulus group) corresponds to or matches the target descriptor, or a true negative, where the subject correctly indicates when the stimulus group does not correspond to or match the target descriptor; or an incorrect response, including: a false negative, where the subject incorrectly indicates that the stimulus group does not correspond to or match the target descriptor, or a false positive, where the subject incorrectly indicates that the stimulus group corresponds to or matches the target descriptor. In other words, if the stimulus group corresponds to or matches the target descriptor, the correct response is to indicate the correspondence or match, and the incorrect response is to (incorrectly) indicate that the stimulus group does not correspondence to or match the target descriptor, and if the stimulus group does not correspond to or match the target descriptor, the correct response is to indicate so, and the incorrect response is to (incorrectly) indicate that the stimulus group corresponds to or matches the target descriptor.

In one embodiment, the subject may be required to respond before a next stimulus group is presented, i.e., before the stimulus's ISI has elapsed. This time period between the moment the stimulus group is presented and the onset of the subsequent stimulus group is referred to as the window of response, and may be modified per trial. In other words, the response window is the duration of the stimulus presentation plus the following ISI time.

In some embodiments, an indication, e.g., an audible or visual indication, may be provided to the subject indicating whether the subject responded correctly, i.e., indicating the correctness or incorrectness of the subject's response. In some embodiments, indicating whether the subject responded correctly may include rewarding the subject if a specified level of success is achieved, or penalizing the subject if a specified level of failure is achieved, where the rewarding and penalizing may each include one or more of: auditory feedback, visual feedback, point modification, or change in bonus status. Of course, any types of indication may be used as desired, e.g., tokens, graphical images, animation, audible rewards, e.g., tunes, etc.

The duration and/or the ISI may be adjusted based on the above determining using an adaptive procedure. For example, if the subject achieves some specified level of success, the duration and/or ISI may be decreased, thereby increasing the difficulty of the task. Conversely, if the subject has achieved some specified level of failure (or failed to achieve a (possibly different) level of success, the duration and/or ISI may be increased, thereby decreasing the difficulty of the task. In preferred embodiments, adjusting the duration and/or the ISI may be performed using an adaptive procedure, e.g., a maximum likelihood procedure. For example, the maximum likelihood procedure may be or include a QUEST (quick estimation by sequential testing) threshold procedure, or a ZEST (zippy estimation by sequential testing) threshold procedure, described below, whereby threshold values for the stimulus duration and/or ISI (or more generally, the stimulus intensity) may be determined based on the subject's performance. In preferred embodiments, a continuous performance maximum likelihood procedure may be used, e.g., continuous performance ZEST or continuous performance QUEST. However, it should be noted that in various embodiments, any adaptive procedure may be used as desired.

In some embodiments, adjusting the stimulus duration and/or ISI may include adjusting the stimulus duration and/or ISI to approach and substantially maintain a specified success rate for the subject, e.g., using a single stair maximum continuous performance likelihood procedure.

In one embodiment, the above presenting the target descriptor, presenting the continuous sequence of stimulus groups, and processing each stimulus group, including the requiring, determining, and adjusting for each stimulus group may compose a session. For each session, an initial value of the duration and/or the ISI and a final value of the duration and/or the ISI may be averaged to determine the initial value of the duration and/or the ISI for the next session. In some embodiments, for each session, reaction times (of the subject) may be averaged for each stimulus group in the continuous sequence of stimulus groups to determine a minimum value for the duration and/or the ISI for the next session, where each reaction time includes a respective delay between the presentation of each stimulus group and the subject's response to the stimulus group. In some embodiments, at the end of each session a reward may be presented, e.g., visually and/or audibly.

In some embodiments, the method may further include performing an initial session prior to performing the above method elements, where the initial session includes the above method elements, but where, in performing the processing of the stimulus group in the initial session, neither the duration nor the ISI is adjusted.

In one embodiment, each stimulus group presentation and corresponding subject response may compose a trial. The method may further include for each trial, recording one or more of: the target descriptor, the stimulus group, whether or not the stimulus group corresponds to the target descriptor, the duration, the ISI, the subject's response, the correctness or incorrectness of the subject's response, the reaction time for the trial, or statistical measures for the adaptive procedure, e.g., the continuous performance maximum likelihood procedure.

The above presenting the target descriptor, presenting the continuous sequence of stimulus groups, and processing each stimulus group, including the requiring, determining, and adjusting for each stimulus group, may be iteratively performed to improve the cognition of the subject, e.g., presenting various stimulus group sequences (stimulus streams), and adjusting the stimulus presentation (e.g., duration and/or ISI) to increase or decrease the task difficulty based on the subject's responses. In some embodiments, the repeating of these method elements may be terminated if the subject responds incorrectly a specified number of times consecutively, e.g., 5 times in a row.

In some embodiments, the above presenting the target descriptor, presenting the continuous sequence of stimulus groups, and processing each stimulus group, including the requiring, determining, and adjusting for each stimulus group, may be performed under a specified condition, where the condition specifies one or more aspects or attributes or aspects of the presenting the continuous sequence(s) of stimulus groups. Moreover, the method may further include performing the repeating a plurality of times, i.e., iteratively, where each iteration is performed under a respective condition. In other words, not only may multiple continuous sequences of stimulus groups be presented (the above repeating), but a plurality of such multiple presentations may be performed as well, each under a respective condition.

As noted above, in some embodiments, the conditions may become more difficult as the subject progresses through the exercise. For example, various conditions or types of target descriptors may correspond to different progressive levels in the exercise, through which the subject may progress as the exercise or task is performed. For example, more difficult words, more complex target descriptors, shorter presentation times, etc., may be used to define blocks or levels of increasing difficulty.

Moreover, the method may include various continuous performance exercises or tasks, where different types or categories of stimuli may be used.

As noted above, in some embodiments, the exercise or task may be presented in a series of sessions. In one embodiment, a session may be a continuous series of trials of the exercise performed for a fixed amount of time with all parameters except the duration/ISI held fixed, and where the duration/ISI may be tuned using an adaptive procedure, e.g., a continuous performance maximum likelihood procedure, e.g., a single stair continuous performance ZEST procedure, described below in more detail. In one embodiment, the initial presentation duration (and thus the ISI duration) may be set by default at some value (˜1 sec), and subsequent duration values may be computed using the ZEST algorithm after each stimulus presentation, with the user's performance poised to converge to the 80%-90% performance level to keep the subjects engaged in the task. In one exemplary embodiment, the session may be initially run for 90 seconds with a 25-75% target frequency, with a reward, currently in the form of a scenic picture, presented at the end of a session. A screen summarizing the subject's performance on the task over the past sessions may be displayed. If the subject makes some predetermined number of consecutive incorrect responses (e.g., 5 or 6 in a row), the session may be terminated.

As indicated above, the subject's response to each trial, i.e. presentation of a stimulus group, may be evaluated to determine the value to feed into the adaptive procedure, e.g., the ZEST algorithm. In addition to a true positive or “hit”, corresponding to a correct response, and false positive or “miss”, corresponding to incorrect responses, the subject's ability to inhibit response to foils (or to correctly indicate non-targets) may also be awarded as correct response. In one embodiment, starting and final values of the ZEST stair may be averaged and used as the starting value for subsequent session. The reaction times for all true positives throughout an entire session may be recorded and averaged, and used as the minimum or floor for the ZEST procedure. As also noted above, in some embodiments, the first session of each exercise is played without employing the ZEST progression, i.e., without adjusting the duration or ISI.

Thus, in various embodiments, the method may include one or more tasks or exercises where a target descriptor is presented, after which a single stream of stimuli is presented, i.e., where the stimulus groups each include a single stimulus, and the user indicates when a stimulus group matches or corresponds to the specified target descriptor(s). Note, however, that in other embodiments, multiple stimulus streams may be used (i.e., where each stimulus group in the sequence of stimulus groups includes a plurality of stimuli), and/or multiple target descriptors may be used. Moreover, as noted above, in some embodiments, other or multiple modes (auditory, textual (orthographic), pictorial, etc.) may be used, e.g., for the target descriptors and/or the stimuli.

Thus, in various embodiments, the method may include one or more tasks or exercises where the target descriptor is presented, after which a continuous sequence of stimulus groups, e.g., a stream of stimuli is presented, and the subject indicates when the stimuli match or correspond to the specified target descriptor(s).

In some embodiments, the exercise may include a combination of various continuous performance tasks. In some embodiments, the repeating of the method elements, and/or the performing the repeating of the method elements a plurality of times, may occur a specified number of times each day, for a specified number of days. In other words, the subject may perform a plurality of sessions each day over a period of days, e.g., for 6 months, to improve cognition.

Threshold Determination

As indicated above, the duration and/or ISI may be adjusted using an adaptive procedure. For example, in some embodiments, the duration and/or ISI may be adjusted using a maximum likelihood procedure. Such procedures may be used to modify or set an adjustable attribute (or combination of attributes) of a presented stimulus, whereby trials in the task or exercise may be made more or less difficult. Such an adjustable parameter is generally referred to as a stimulus intensity, and the maximum likelihood procedure is used to determine a stimulus threshold, which is the value of the stimulus intensity at which the subject achieves a specified level of success, e.g., 0.9, corresponding to a 90% success rate. There are various approaches whereby such thresholds may be assessed or determined, such as, for example, the well known QUEST (Quick Estimation by Sequential Testing) threshold method, which is an adaptive psychometric procedure for use in psychophysical experiments, or a related method, referred to as the ZEST (Zippy Estimation by Sequential Testing) procedure or method, among others.

Exercise based threshold determination may be designed to assess a subject's threshold with respect to stimuli on a given exercise, and can be used to adjust stimulus presentation to achieve and maintain a desired success rate for the subject, e.g., with respect to a particular exercise, task, and/or condition. In preferred embodiments of the exercises and tasks described herein, the stimulus intensity is the duration and/or ISI of the presented stimuli. In other words, the progressions (successive modifications or adjustments of presentation parameters, e.g., duration and/or ISI) in the exercise may be calculated using an adaptive procedure, e.g., a maximum likelihood procedure, e.g., the ZEST procedure. For example, for each trial, a likelihood function may be calculated (based on the subject's response) to determine the next best guess of the true threshold. This estimate may be used (and possibly displayed) in the next trial. Based on the trial outcome and all previous trials the next best guess may be calculated and used in the presentation of stimuli for the next trial (and possibly displayed). As the probability function narrows and the standard deviation decreases, the estimate of true threshold approaches the true value—e.g., achieving a “good enough” threshold value. Note that the ZEST procedure is a modification of the QUEST procedure—the ZEST procedure uses the mean while the QUEST procedure uses the mode. In preferred embodiments, when training, a single stair ZEST procedure may be used, e.g., with a threshold level of 85%, although other values may be used as desired.

In preferred embodiments, the maximum likelihood procedure may be a continuous performance maximum likelihood procedure (as opposed to a discrete performance maximum likelihood procedure), such as a continuous performance ZEST procedure. One such a procedure, referred to as a continuous performance task (CPT), is described below.

The Continuous Performance Task (CPT) is a neuropsychological task or exercise that consists of a series of stimulus groups presented one after another. The subject may be instructed to attend to a particular stimulus or a category of stimuli and respond to the presented stimulus group. The initial duration of CPT, e.g., the initial value of the presentation time the stimulus groups, may differ from task to task.

Continuous Performance Tasks have traditionally been conducted where the stimulus Onset Asynchrony (SOA) time, e.g., the duration plus the ISI, is fixed throughout a session or is only altered after a block of stimuli is presented. Because the appropriate SOA time may differ greatly depending on: 1) the nature of the task; 2) the type of stimulus set used; and 3) the ability of the subject, a progression algorithm that optimizes the training experience is desired, especially when the training of speed of processing is crucial. For example, if the SOA time is too long, the task will not challenge the subjects and thus not engage them. If the SOA time is too short, subjects may find the task frustrating and the task may lack training value. Thus, a new SOA time progression scheme is presented herein that optimizes training experience by changing the SOA from trial to trial using an adaptive procedure, e.g., based on a maximum likelihood method. This scheme is used in preferred embodiments of the continuous performance cognitive training exercises described above.

In one embodiment, the progression scheme employed is based on the ZEST (Zippy Estimation by Sequential Testing) Bayesian adaptive estimator method, which is a probabilistic procedure where a prior estimate value of a variable is updated sequentially via a likelihood function that contains all the previous trials' information, yielding a posterior estimate value that encompasses all the data generated so far from the initial assumptions to all the user's responses. In other embodiments, any other adaptive procedures may be used as desired, e.g., QUEST.

Other features and advantages of the present invention will become apparent upon study of the remaining portions of the specification and drawings.

### BRIEF DESCRIPTION OF THE DRAWINGS

FIG. 1 is a block diagram of a computer system for executing a program according to some embodiments of the present invention;

FIG. 2 is a block diagram of a computer network for executing a program according to some embodiments of the present invention;

FIG. 3 is a high-level flowchart of one embodiment of a method for cognitive training using stimulus streams with target descriptors, according to one embodiment;

FIG. 4 illustrates an exemplary graphical user interface (GUI) presenting task instructions and a target descriptor for matching a word to an object, according to one embodiment;

FIG. 5 illustrates an exemplary GUI presenting a visual stimulus for the task of FIG. 4, according to one embodiment;

FIG. 6 illustrates an exemplary graphical user interface (GUI) presenting task instructions and a target descriptor for matching a word to a category, according to one embodiment;

FIG. 7 illustrates an exemplary GUI presenting a visual stimulus for the task of FIG. 6, according to one embodiment;

FIG. 8 illustrates an exemplary graphical user interface (GUI) presenting task instructions and a target descriptor for matching a word to an attribute, according to one embodiment;

FIG. 9 illustrates an exemplary GUI presenting a visual stimulus for the task of FIG. 8, according to one embodiment;

FIG. 10 illustrates an exemplary psychometric function based on a cumulative Gaussian distribution for a detection (yes/no) task with a 5% lapsing rate, and a target performance rate of 85%, according to one embodiment;

FIG. 11 illustrates an exemplary probability density function with initial intensity estimate x=5, according to one embodiment;

FIG. 12 illustrates exemplary likelihood functions with T=5 and an 85% correct rate, according to one embodiment;

FIG. 13 illustrates an exemplary prior probability density function (P.D.F.) superimposed with exemplary likelihood functions, according to one embodiment; and

FIG. 14 illustrates an exemplary posterior P.D.F. with Yes/No response, according to one embodiment.

### DETAILED DESCRIPTION

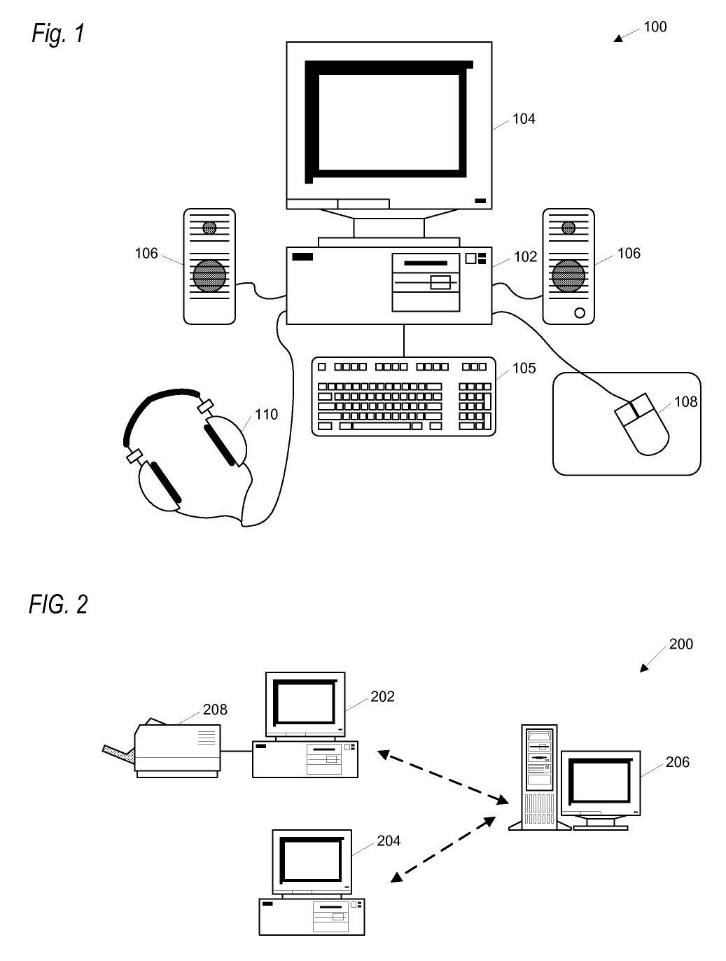

Referring to FIG. 1, a computer system 100 is shown for executing a computer program to train, or retrain an individual according to the present invention to enhance cognition, where the term “cognition” refers to the speed, accuracy and reliability of processing of information, and attention and/or memory, and where the term “attention” refers to the facilitation of a target and/or suppression of a non-target, e.g., over a given spatial extent, object-specific area, or time window, e.g., with respect to one or more stimulus streams, such as in a continuous performance exercise, and using a continuous performance maximum likelihood procedure to adjust presentation of the stimuli based on the subject's performance. As shown, in this embodiment, the computer system 100 contains a computer 102, having a CPU, memory, hard disk and CD ROM drive (not shown), attached to a monitor 104. The monitor 104 provides visual prompting and feedback to the subject during execution of the computer program. Attached to the computer 102 are a keyboard 105, speakers 106, a mouse 108, and headphones 110. In some embodiments, the speakers 106 and the headphones 110 may provide auditory prompting, stimuli, and feedback to the subject during execution of the computer program. The mouse 108 allows the subject to navigate through the computer program, and to select particular responses after visual or auditory prompting by the computer program. The keyboard 105 allows an instructor to enter alphanumeric information about the subject into the computer 102, and/or response by the subject. Although a number of different computer platforms are applicable to the present invention, embodiments of the present invention execute on either IBM compatible computers or Macintosh computers, or similarly configured computing devices such as set top boxes, PDA's, gaming consoles, etc.

Now referring to FIG. 2, a computer network 200 is shown. The computer network 200 contains computers 202, 204, similar to that described above with reference to FIG. 1, connected to a server 206. The connection between the computers 202, 204 and the server 206 can be made via a local area network (LAN), a wide area network (WAN), or via modem connections, directly or through the Internet. A printer 208 is shown connected to the computer 202 to illustrate that a subject can print out reports associated with the computer program of the present invention. The computer network 200 allows information such as test scores, game statistics, and other subject information to flow from a subject's computer 202, 204 to a server 206. An administrator can review the information and can then download configuration and control information pertaining to a particular subject, back to the subject's computer 202, 204.

Embodiments of the computer-based exercises or tasks described herein may operate to renormalize and improve the ability of the nervous system to perceive, process, and remember, information via continuous performance using stimulus streams and a continuous performance maximum likelihood procedure. This may be achieved by having subjects perform any of various tasks using stimuli under conditions of high engagement/stimulation and under high reward for correct performance in order to encourage renormalization of cognition, and memory.

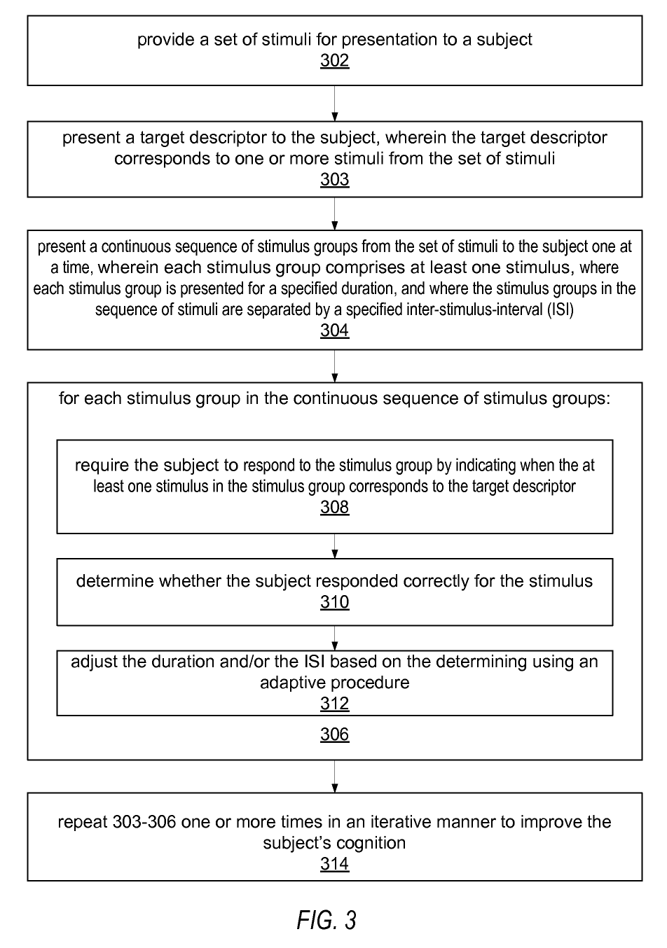

FIG. 3—Flowchart of a Method for Cognitive Training Using Continuous Performance

FIG. 3 is a high-level flowchart of one embodiment of a method for cognitive training using continuous stimuli, i.e., one or more stimulus streams, and a continuous performance maximum likelihood procedure. More specifically, the method utilizes a computing device to present one or more stimulus streams, and to record responses from the subject. A primary goal of the method is to train the subjects to maintain attention and vigilance on a specific target. The subject is to respond positively when presented with the desired target, and to respond negatively or refrain from indicating when presented with non-target stimuli. As the subject progresses through the exercise, the presentation of the stimuli may be shortened to require quicker and more accurate response from the subject, specifically, via an adaptive procedure, such as, for example, a continuous performance maximum likelihood procedure. Successful completion of the task may improve the concentration and focus system as well as elevating the speed of processing.

The method may be used in the context of any of a variety of cognitive training exercises or tasks using continuous performance with stimulus streams, examples of which are described below. It should be noted that in various embodiments, some of the method elements may be performed concurrently, in a different order than shown, or may be omitted. Additional method elements may also be performed as desired. As shown, the method may be performed as follows:

In 302, a set (or sets) of stimuli may be provided for presentation to the subject. For example, the stimuli may be stored on a memory medium of the computing device, on a memory medium coupled to the computing device, e.g., over a network, etc. The stimuli may include various types of stimuli, e.g., visual stimuli, e.g., orthographic words or numbers, pictorial stimuli, e.g., geometric shapes with various attributes, and so forth, as well as auditory stimuli, e.g., spoken words or phrases (via human recordings or synthesized speech), sounds, etc., as desired. In some embodiments, stimuli may be provided in multiple different modes, e.g., auditory, orthographic (textual), or pictorial stimuli may be used, among others. Note that as used herein, a “more difficult stimulus” means that in the context of a cognitive training task, the presentation of the stimulus would result in a lower probability of correct response by the subject.

In 303, a target descriptor may be presented to the subject. The target descriptor may be presented visually and/or audibly as desired, and may correspond to one or more stimuli from the set of stimuli. In various embodiments, the target descriptor may specify any of various matching or correspondence criteria, e.g., target correspondence, target attributes, possibly including the absence of attributes, correspondence condition(s), and so forth, indicating to the subject under what conditions the presented stimuli may be considered to correspond or match, e.g., with respect to the target (e.g., in the case of target attributes), or among themselves (e.g., in the case of a target correspondence). In other words, the target descriptor may specify target attributes and/or relationships among the stimuli, or any combination. Various exemplary tasks are described below illustrating exemplary target descriptors, although it should be noted that any types of target descriptors may be used as desired, e.g., arbitrary attributes of arbitrary stimuli.

For example, in some exemplary embodiments, the target descriptor may specify one or more of: a word or object, a category, and/or one or more attributes (or the absence of attributes), among others. For example, in one embodiment of an exemplary word/object matching task, referred to as Task 1 for convenience, the subject is to seek out the target object from a stream of simple objects consisting of the target objects and non-target objects, and indicate matches or correspondences.

Example categories include ““animal”, “clothing”, and “body parts”, among others. Thus, the stimulus “dog” would match or correspond to the category “animal”, but “shoe” would not. In one embodiment of a category matching task, referred to as Task 2 for convenience, the subject is to indicate when a stimulus matches or corresponds to the target category.

In one embodiment of the attribute(s) task, referred to as Task 3 for convenience, the subject is to indicate when a stimulus matches or corresponds to the target attribute(s). Example attributes include “blue figure”, and “one element” (e.g., quantity). Note that the target descriptor may also be compound, e.g., may include multiple objects, categories, attributes (possibly including absence of attributes), and/or correspondence conditions, or any combination of such. For example, an exemplary compound target descriptor may be “not Shrek, green monsters with two shoes”. Thus, a target descriptor may itself include multiple target descriptors.

Other exemplary target descriptors may specify attribute relationships. For example, the target descriptor may specify one or more “identical” or “distinct” attributes, among one or more stimulus attributes, e.g., color, shape, texture, quantity, and the absence or negative of any of these attributes (e.g., “not blue”). Example target attributes include: “identical color”, meaning that all the stimuli in the stimulus group have the same color, and “identical shape and distinct quantity”, meaning that all the stimuli in the stimulus group have the same shape, but each stimulus in the group has a different quantity or number of elements, and so forth.

In 304, a continuous sequence of stimulus groups from the set of stimuli may be presented to the subject one stimulus at a time, where the continuous sequence of stimulus groups includes one or more stimuli corresponding to the target descriptor, and one or more stimuli not corresponding to the target descriptor. The stimuli that do correspond to or match the target descriptor may be referred to as corresponding or matching stimuli, target stimuli, or simply targets. Those that do not match or correspond to the target descriptor may be referred to as “foils”, non-matching or non-corresponding stimuli, non-target stimuli, or simply non-targets.

Each stimulus group may be presented for a specified duration, and the stimulus groups in the continuous sequence of stimulus groups may be separated by a specified inter-stimulus-interval (ISI). Note that in embodiments where each stimulus group includes a single stimulus, the continuous sequence of stimulus groups composes, includes, or corresponds to a continuous stimulus stream, i.e., each stimulus group is simply a single stimulus. Similarly, in embodiments where each stimulus group includes one or more stimuli, the continuous sequence of stimulus groups includes or corresponds to one or more continuous stimulus streams, where the one or more stimuli in each stimulus group are from respective continuous stimulus streams of the one or more continuous stimulus streams. In other words, each stimulus group includes stimuli from respective (one or more) stimulus streams, and the presenting of the sequence of stimulus groups includes presenting the one or more stimulus streams to the subject, with each stimulus stream contributing a respective stimulus for each stimulus group. For example, in an example where three stimulus streams are used, the stimulus group may include a solid blue square, a solid blue circle, and a nested blue triangle, which in this case, would match or correspond with target attributes such as “blue”, “not red”, “not green”, “not open”. Other examples of such stimulus groups are described below.

The particular stimuli presented may be dependent on the particular task being performed. For example, the stimuli for Task 1 may include various words or objects (e.g., presented textually or pictorially), the stimuli for Task 2 may include objects in the class of the category (e.g. for category “animal”, target stimuli may include “dog”, “ant”, etc.) and objects that do not fall into the category (e.g. “box”, “shoes”, etc.), and, following the example above, with the attribute “blue figure”, the stimuli for Task 3 may include a set of geometrical objects (e.g. circle, triangle, square) of various colors (e.g. blue, green, red), where the subject is to respond only when figure of the desired color is shown. Note, however, that in other embodiments, stimuli in accordance with any other types, modes, or categories may be used as desired. Note further that, as mentioned above, in various embodiments, the continuous sequence of stimulus groups may be presented in a specified mode or modes, including one or more of: an auditory mode, wherein each stimulus or stimulus group is presented audibly, a pictorial mode, wherein each stimulus or stimulus group is presented pictorially, or an orthographic mode, wherein each stimulus or stimulus group is presented textually, among others.

As mentioned above, in one embodiment, the at least one stimulus in the stimulus group may include a plurality of stimuli, where the target descriptor includes or specifies one or more relationships of attributes of the plurality of stimuli. Each relationship may include one or more of: identical, where each stimulus of the plurality of stimuli has a common attribute value, or distinct, where each stimulus of the plurality of stimuli has a different attribute value. Moreover, as also indicated above, in some embodiments, the attributes of the plurality of stimuli may include two or more of: color, shape, texture, quantity, absence of a specified color, absence of a specified shape, absence of a specified texture, or absence of a specified quantity, among others. Note that in some embodiments, the target descriptor(s) of 303 may be presented concurrently with the stimulus (i.e., on the same screen or with sound), while in others, the target descriptor(s) may be presented prior to the stimuli.

As FIG. 3 indicates, in 306, each stimulus group in the continuous sequence of stimulus groups may be processed as follows (308-312):

In 308, the subject may be required to respond to the stimulus group by indicating when the stimulus group (i.e., all the stimuli in the stimulus group) corresponds to or matches the target descriptor. In some embodiments, the subject may be required to respond differently when the stimulus group does not match or correspond to the target descriptor, or to refrain from indicating when the stimulus group does not correspond to the target descriptor. Said another way, in these embodiments, when the stimulus group matches or corresponds to the target descriptor, the subject should indicate that this is so. However, when the stimulus group does not match or correspond to the target descriptor, in some embodiments, the subject should indicate so with a different response, while in other embodiments, the subject should refrain from responding. As noted above, “corresponding” or “matching” stimuli, refer to stimuli that correspond to the specified target descriptor(s). Thus, for example, in some embodiments where the subject is to respond positively when the stimulus group matches, and negatively when the stimulus group doesn't match, the subject may press a first key, e.g., an up arrow key, when the stimulus group matches or corresponds to the target descriptor, and may press a second key, e.g., a down arrow key, when the stimulus group doesn't match. Of course, other keys or GUI controls may be used as desired. In embodiments where the subject is to inhibit responses to non-target stimuli, when the stimulus group doesn't match or correspond to the target descriptor, the subject should do nothing.

Thus, in a preferred embodiment, one or more stimulus streams (continuous sequence of stimulus groups) may be presented on a visual display device, e.g., a computer monitor, where the subject may be required to respond when the stimulus group matches or corresponds to the target descriptor, e.g., by pressing the spacebar on the keyboard, clicking on a GUI control, etc.

In 310, a determination may be made as to whether the subject responded correctly for the stimulus group. For example, in some embodiments where the subject is to inhibit responses to non-targets, the subject's response to the stimulus group may include one of: a correct response, including: a true positive, where the subject correctly indicates when the stimulus group (i.e., the at least one stimulus in the stimulus group) corresponds to or matches the target descriptor, or a true negative, where the subject correctly refrains from indicating when the stimulus group does not correspond to or match the target descriptor; or an incorrect response, including: a false negative, where the subject (incorrectly) fails to indicate when the stimulus group corresponds to or matches the target descriptor, or a false positive, where the subject incorrectly indicates that the stimulus group corresponds to or matches the target descriptor. In other words, in these embodiments, if the stimulus group corresponds to or matches the target descriptor, the correct response is to indicate the correspondence or match, and the incorrect response is to fail to indicate the correspondence or match, and if the stimulus group does not correspond to or match the target descriptor, the correct response is to refrain from indicating a correspondence or match, and the incorrect response is to (incorrectly) indicate that the stimulus group corresponds to or matches the target descriptor.

In some embodiments where the subject is to respond by indicating both targets and non-targets, the subject's response to the stimulus group may include one of: a correct response, including: a true positive, where the subject correctly indicates when the stimulus group (i.e., the at least one stimulus in the stimulus group) corresponds to or matches the target descriptor, or a true negative, where the subject correctly indicates when the stimulus group does not correspond to or match the target descriptor; or an incorrect response, including: a false negative, where the subject incorrectly indicates that the stimulus group does not correspond to or match the target descriptor, or a false positive, where the subject incorrectly indicates that the stimulus group corresponds to or matches the target descriptor. In other words, if the stimulus group corresponds to or matches the target descriptor, the correct response is to indicate the correspondence or match, and the incorrect response is to (incorrectly) indicate that the stimulus group does not correspondence to or match the target descriptor, and if the stimulus group does not correspond to or match the target descriptor, the correct response is to indicate so, and the incorrect response is to (incorrectly) indicate that the stimulus group corresponds to or matches the target descriptor.

In one embodiment, the subject may be required to respond before a next stimulus group is presented, i.e., before the stimulus's ISI has elapsed. This time period between the moment the stimulus group is presented and the onset of the subsequent stimulus group is referred to as the window of response, and may be modified per trial. In other words, the response window is the duration of the stimulus presentation plus the following ISI time.

In some embodiments, an indication, e.g., an audible or visual indication, may be provided to the subject indicating whether the subject responded correctly, i.e., indicating the correctness or incorrectness of the subject's response. For example, in one embodiment, a “ding” or a “thunk” (or corresponding equivalents) may be played to indicate correctness or incorrectness, respectively. In some embodiments, indicating whether the subject responded correctly may include rewarding the subject if a specified level of success is achieved, or penalizing the subject if a specified level of failure is achieved, where the rewarding and penalizing may each include one or more of: auditory feedback, visual feedback, point modification, or change in bonus status. Of course, any types of indication may be used as desired, e.g., tokens, graphical images, animation, audible rewards, e.g., tunes, etc.

In 312, the duration and/or the ISI may be adjusted based on the determining of 308, using an adaptive procedure. For example, if the subject achieves some specified level of success, the duration and/or ISI may be decreased, thereby increasing the difficulty of the task. Conversely, if the subject has achieved some specified level of failure (or failed to achieve a (possibly different) level of success, the duration and/or ISI may be increased, thereby decreasing the difficulty of the task. In preferred embodiments, the adaptive procedure may comprise a maximum likelihood procedure. For example, the maximum likelihood procedure may be or include a QUEST (quick estimation by sequential testing) threshold procedure, or a ZEST (zippy estimation by sequential testing) threshold procedure, described below, whereby threshold values for the stimulus duration and/or ISI (or more generally, the stimulus intensity) may be determined based on the subject's performance. In preferred embodiments, a continuous performance maximum likelihood procedure may be used, e.g., continuous performance ZEST or continuous performance QUEST. However, it should be noted that in various embodiments, any adaptive procedure may be used as desired.

In some embodiments, adjusting the stimulus duration and/or ISI may include adjusting the stimulus duration and/or ISI to approach and substantially maintain a specified success rate for the subject, e.g., using a single stair maximum continuous performance likelihood procedure, also described below.

In one embodiment, the method elements 303-306 (including 308-312) may compose a session. In other words, the above presenting the target descriptor (303), presenting the continuous sequence of stimulus groups (304), and processing each stimulus group (306), including the requiring (308), determining (310), and adjusting (312) for each stimulus group may compose a session. For each session, an initial value of the duration and/or the ISI and a final value of the duration and/or the ISI may be averaged to determine the initial value of the duration and/or the ISI for the next session. In some embodiments, for each session, reaction times (of the subject) may be averaged for each stimulus group in the continuous sequence of stimulus groups to determine a minimum value for the duration and/or the ISI for the next session, where each reaction time includes a respective delay between the presentation of each stimulus group and the subject's response to the stimulus group. In some embodiments, at the end of each session a reward may be presented, e.g., visually and/or audibly.

In some embodiments, the method may further include performing an initial session prior to performing 303-306, where the initial session includes 303-306, but where, in performing 306 in the initial session, neither the duration nor the ISI is adjusted.

In one embodiment, each stimulus group presentation and corresponding subject response may compose a trial. The method may further include for each trial, recording one or more of: the target descriptor, the stimulus group, whether or not the stimulus group corresponds to the target descriptor, the duration, the ISI, the subject's response, the correctness or incorrectness of the subject's response, the reaction time for the trial, or statistical measures for the adaptive procedure, e.g., the continuous performance maximum likelihood procedure, described in more detail below.

In 314, the method elements 303-306 (including 308-312) may be repeated one or more times in an iterative manner to improve the cognition of the subject. In other words, the above presenting the target descriptor (303), presenting the continuous sequence of stimulus groups (304), and processing each stimulus group (306), including the requiring (308), determining (310), and adjusting (312) for each stimulus group, may be iteratively performed to improve the cognition of the subject, e.g., presenting various stimulus group sequences (stimulus streams), and adjusting the stimulus presentation (e.g., duration and/or ISI) to increase or decrease the task difficulty based on the subject's responses. In some embodiments, the repeating of 314 may be terminated if the subject responds incorrectly a specified number of times consecutively, e.g., 5 times in a row.

In some embodiments, the above method elements 303-306 may be performed under a specified condition, where the condition specifies one or more aspects or attributes or aspects of the presenting the continuous sequence(s) of stimulus groups. Moreover, the method may further include performing the repeating of 314 a plurality of times, i.e., iteratively, where each iteration is performed under a respective condition. In other words, not only may multiple continuous sequences of stimulus groups be presented (the repeating of 314), but a plurality of such multiple presentations may be performed as well, each under a respective condition.

In one embodiment, each condition may specify one or more of: session length, e.g., length of the continuous sequence of stimulus groups, and/or length of time of said presenting the continuous sequence of stimulus groups, correspondence frequency, e.g., a ratio of stimulus groups in which all the stimuli correspond to the target descriptor to stimulus groups in which all the stimuli do not correspond to the target descriptor (may be shortened or randomized to test subject's ability to focus attention and recall targets in various frequencies), or target/foil confusability, e.g., a degree to which stimuli that correspond to the target descriptor are similar to stimuli that do not correspond, (e.g. target: bean, foil: been). In other embodiments, other attributes of the stimulus presentation may be specified as desired.

As noted above, in some embodiments, the conditions may become more difficult as the subject progresses through the exercise. For example, various conditions or types of target descriptors may correspond to different progressive levels in the exercise, through which the subject may progress as the exercise or task is performed. For example, more difficult words, more complex target descriptors, shorter presentation times, etc., may be used to define blocks or levels of increasing difficulty.

Moreover, the method may include various continuous performance exercises or tasks, where different types or categories of stimuli and different types of target descriptors may be used, exemplary embodiments of which are described below.

Exemplary Embodiments of Continuous Performance Tasks

The following describes various exemplary embodiments of continuous performance tasks, following the method of FIG. 3, described above, where once the session begins, a continuous stream of stimuli is presented, e.g., in the form of a sequence of stimulus groups, each stimulus group composed of one or more stimuli, e.g., figures or shapes, words, and/or sounds, and where the subject is required to respond, e.g., by pressing the spacebar on the keyboard (or other indication means), when the stimulus group matches or corresponds to the criteria (the target descriptor) described at the beginning of the session (or during the session).

Note that the tasks described below are exemplary only, and are not intended to limit the exercise to any particular set of tasks or types of stimuli.

Task 1: Match to Word/Object

In one exemplary task (referred to herein as Task 1 for convenience), the subject may be given a target word or object at the start of a session in one of 3 modes (auditory, image, text), i.e., the target descriptor may appear on the screen or be presented via speakers or headphones. In some embodiments, a session begins with the displaying of the instruction for the exercise or task, e.g., explaining to the subject what to expect, and what is expected of the subject in the exercise or task, along with the target descriptor. The subject may then provide an observing response to indicate they are attentive and ready to perform the task, e.g., press a key on the keyboard (e.g., a spacebar) or use other indication means, at which time the instruction and target may be removed from the exercise interface. Following a delay, e.g., of 200 ms, a stream of stimuli may be presented continuously one after another, e.g., on the computer screen, speakers, or over the headphones, and the subject may be required to respond when the target word or pictorial representation of the word or object is shown among the stream of stimuli being continuously presented, e.g., by pressing a key on the keyboard, e.g., the spacebar. The task may be made more difficult by shortening both the duration of the stream presentation and the inter-stimulus interval (ISI) between the successive stimulus presentations. Initial values for the duration and ISI of the presentation may be randomly chosen, determined based on prior studies, or based on past performances of the exercise, among other initialization techniques.

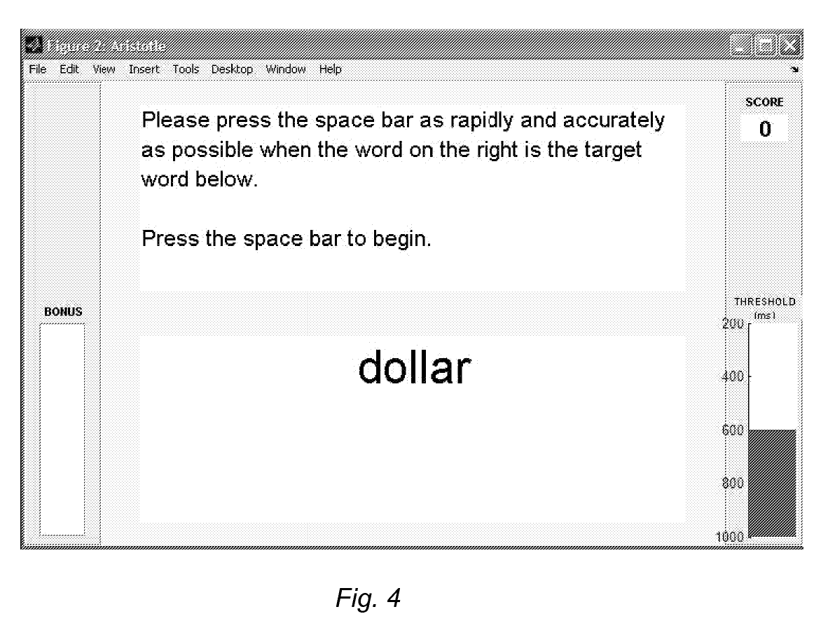

FIG. 4 is a screenshot of an exemplary GUI for Task 1, according to one embodiment. As FIG. 4 shows, in this embodiment, instructions are presented indicating that in this task or exercise, the subject is to press the space bar as quickly and accurately as possible when a stimulus (e.g., a word) is displayed that matches or corresponds to the target descriptor. As may be seen, the GUI also displays the target descriptor, in this case, the word “dollar”.

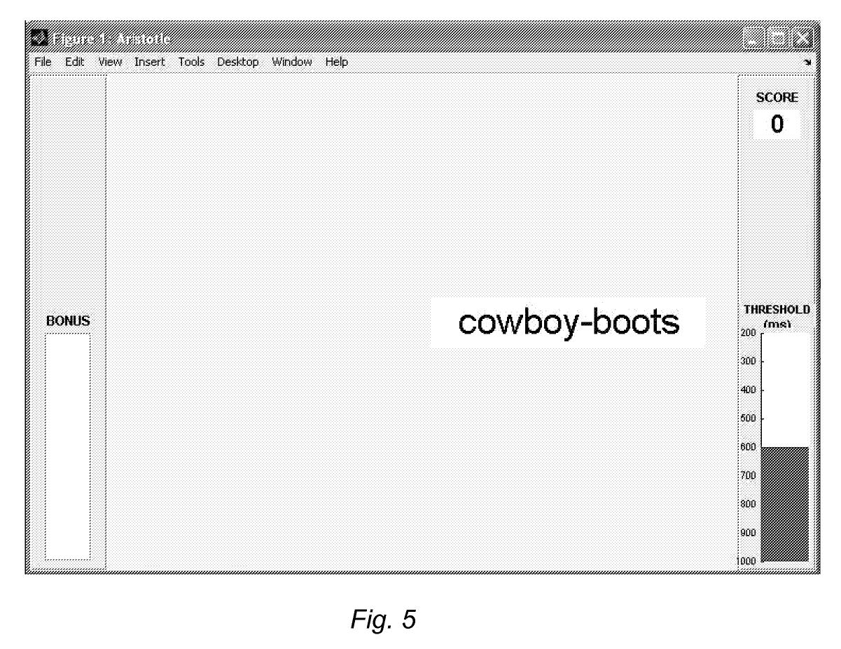

FIG. 5 is a screenshot of an exemplary GUI according to Task 1, described above, where the stimulus presented (in a visual or display field of the GUI) is the word “cowboy-boots”, which clearly does not correspond to or match the target descriptor “dollar”, and so the subject should not respond.

As noted above, in some embodiments, the exercise or task may be presented in a series of sessions. In one embodiment, a session may be a continuous series of trials of the exercise performed for a fixed amount of time with all parameters except the duration/ISI held fixed, and where the duration/ISI may be tuned using an adaptive procedure, e.g., a continuous performance maximum likelihood procedure, e.g., a single stair continuous performance ZEST procedure, described below in more detail. In one embodiment, the duration and ISI may be co-varied together in a fixed predetermined ratio. That is, a subject may move through the task in a session along a one-dimensional subspace of the 2-dimensional duration/ISI parameter space based on the subject's responses to all previous trials, e.g., calculated by the ZEST algorithm. In one embodiment, the initial presentation duration (and thus the ISI duration) may be set by default at some value (˜1 sec), and subsequent duration values may be computed using the ZEST algorithm after each stimulus presentation, with the user's performance poised to converge to the 80%-90% performance level to keep the subjects engaged in the task. In one exemplary embodiment, the session may be initially run for 90 seconds with a 25-75% target frequency, with a reward, currently in the form of a scenic picture, presented at the end of a session. A screen summarizing the subject's performance on the task over the past sessions may be displayed. If the subject makes some predetermined number of consecutive incorrect responses (e.g., 5 or 6 in a row), the session may be terminated.

As indicated above, the subject's response to each trial, i.e. presentation of a stimulus group, may be evaluated to determine the value to feed into the adaptive procedure, e.g., the ZEST algorithm. In addition to a true positive or “hit”, corresponding to a correct response, and false positive or “miss”, corresponding to incorrect responses, the subject's ability to inhibit response to foils (or to correctly indicate non-targets) may also be awarded as correct response. In one embodiment, starting and final values of the ZEST stair may be averaged and used as the starting value for subsequent session. The reaction times for all true positives throughout an entire session may be recorded and averaged, and used as the minimum or floor for the ZEST procedure. As also noted above, in some embodiments, the first session of each exercise is played without employing the ZEST progression, i.e., without adjusting the duration or ISI.

In one embodiment, a block may refer to a series of sessions of an exercise with a specified target/foil set and secondary parameter values (e.g., session time, target frequency). The purpose of the block is to determine the subjects' threshold duration/ISI time under the same condition. As noted above, in one embodiment, the average of the initial and final values of the duration/ISI from the previous session may be used as the initial duration/ISI time for the current session. The subject may continue sessions in a block in this manner until performance plateaus, i.e., until no significant improvement is observed in the final duration/ISI performance from session to session for a predetermined consecutive number of sessions, at which time, the block may be exited. The subject may also exit a block when a specified number of sessions have been performed. The subject may exit the task when there are no further blocks to perform.

Thus, in various embodiments, the method may include one or more tasks or exercises where a target descriptor is presented, after which a single stream of stimuli is presented, i.e., where the stimulus groups each include a single stimulus, and the user indicates when a stimulus group matches or corresponds to the specified target descriptor(s). Note, however, that in other embodiments, multiple stimulus streams may be used (i.e., where each stimulus group in the sequence of stimulus groups includes a plurality of stimuli), and/or multiple target descriptors may be used. Moreover, as noted above, in some embodiments, other or multiple modes (auditory, textual (orthographic), pictorial, etc.) may be used, e.g., for the target descriptors and/or the stimuli.

Task 2: Match to Category

In another exemplary task (referred to herein as Task 2 for convenience), the target descriptor may specify a target category. As with Task 1, the target descriptor may appear on the screen (or may be presented via speakers or headphones), and the subject may press a key on the keyboard to begin. Stimuli (e.g., words or figures) may appear in a continuous stream on the screen. The task is to press the spacebar every time a stimulus group appears that matches the target category. Example target categories include: “flowers”, “words that begin with ‘p’”, and so forth.

Task 2 may operate in a similar manner to the example Task 1 described above, where the subject performs trials in a series of sessions organized by blocks, each block corresponding to a condition, but where the subject attempts to indicate matches or correspondence between stimuli and a target category. As with Task 1, in some embodiments, the subject may be provided the target descriptor (category) at the start of a session in one of three modes (auditory, image, text), i.e., the target descriptor may appear on the screen or may be presented via speakers or headphones. In some embodiments, a session begins with the displaying of the instruction for the exercise or task, e.g., explaining to the subject what to expect, and what is expected of the subject in the exercise or task, along with the target descriptor.

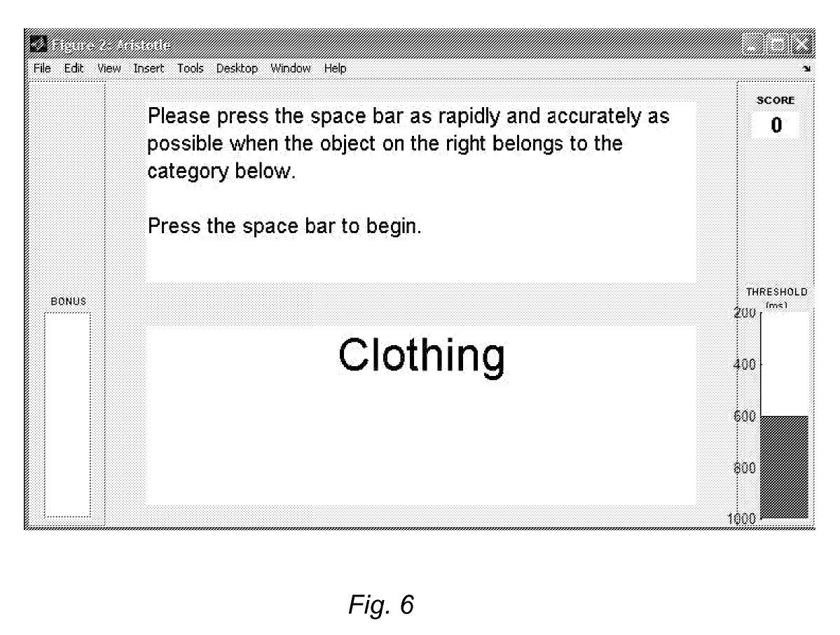

FIG. 6 is a screenshot of an exemplary GUI for the example Task 2, according to one embodiment. As FIG. 6 shows, in this embodiment, instructions are presented indicating that in this task or exercise, the subject is to press the space bar as quickly and accurately as possible when a presented stimulus (object), e.g., a stimulus group including one or more stimuli, belongs to the target category. As may be seen, the GUI also displays the target descriptor (target category), in this case: “Clothing”, meaning that the subject is to respond positively (e.g., hitting the space bar) whenever the stimulus is an article of clothing.

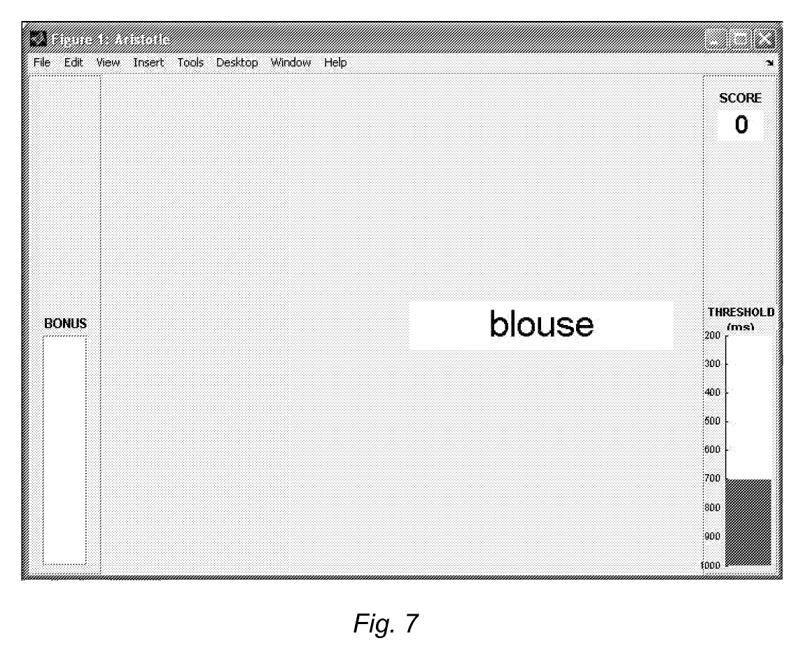

FIG. 7 illustrates another screenshot of an exemplary GUI for Task 2, following the example of FIG. 6. In this example, the stimulus is the word “blouse”, which is clearly in the category “clothing”. Thus, the subject should indicate a match or correspondence.

Task 3: Match to Attributes

In another exemplary task (referred to herein as Task 3 for convenience), the target descriptor may specify one or more target attributes. This example task may be similar to Task 1, but may utilize geometrical figures as stimuli, where the target attributes are properties of some of the figures (stimuli). In some embodiments, the subject may be presented with one or more attributes with targeted (an possibly non-targeted) values at the start of a session. Once the session begins, a stream of figures may be presented on the computer screen, and the subject may be required to respond when the figures possess attributes with values equal to that of the target attribute values, e.g., by pressing a key on the keyboard.

In preferred embodiments, the task may be made more difficult by shortening the duration of the stream presentation and/or the inter-stimulus interval (ISI) between the successive stimulus presentations, as with the above tasks. However, in other embodiments, other parameters may be adjusted as desired, e.g., the number of attributes to test, the number of non-testing attributes to fix, and the number of test-attributes set to non-targets, among others.

Task 3 may operate in a similar manner to the example tasks described above, where the subject performs trials in a series of sessions organized by blocks, each block corresponding to a condition, but where the subject attempts to indicate matches or correspondence between stimuli and one or more target attributes. As with Tasks 1 and 2, in some embodiments, the subject may be provided the target descriptor (attributes) at the start of a session in one of three modes (auditory, image, text), i.e., the target descriptor may appear on the screen or may be presented via speakers or headphones. In some embodiments, a session begins with the displaying of the instruction for the exercise or task, e.g., explaining to the subject what to expect, and what is expected of the subject in the exercise or task, along with the target descriptor.

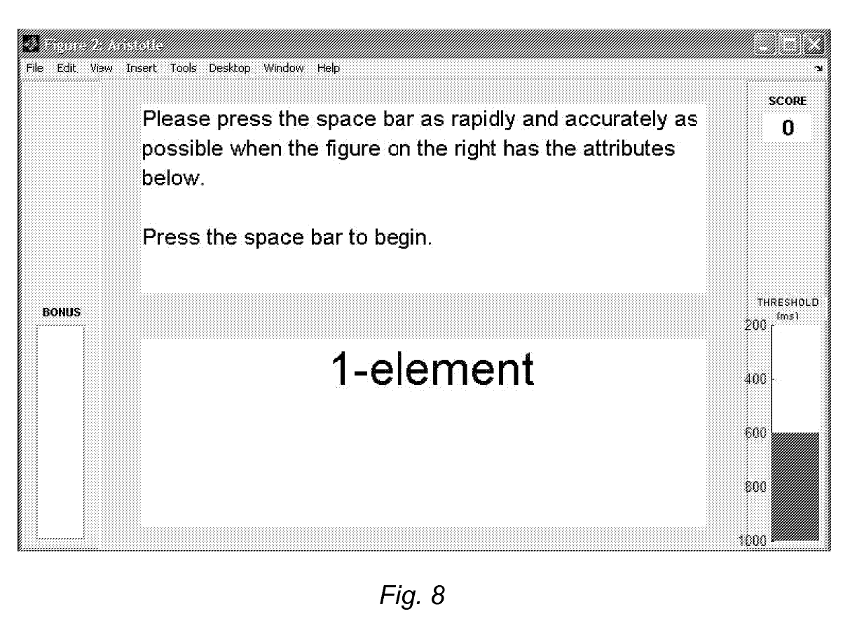

FIG. 8 is a screenshot of an exemplary GUI for the example Task 3, according to one embodiment. As FIG. 8 shows, in this embodiment, instructions are presented indicating that in this task or exercise, the subject is to press the space bar as quickly and accurately as possible when a presented stimulus group (figure) has the target attribute(s). As may be seen, the GUI also displays the target descriptor (target attribute(s)), in this case: “1-element”, meaning that the subject is to respond positively (e.g., hitting the space bar) whenever the stimulus is a single element figure.

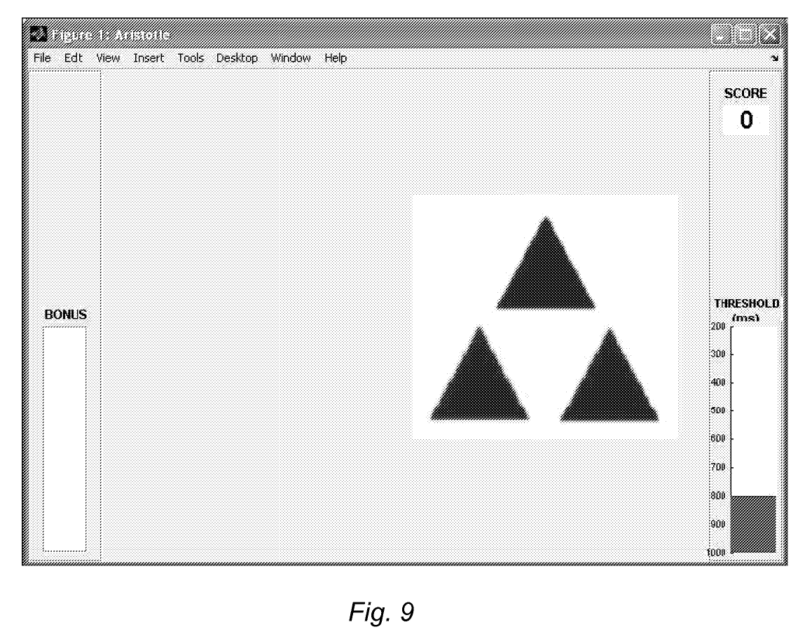

FIG. 9 illustrates another screenshot of an exemplary GUI for Task 3, following the example of FIG. 8. In this example, the stimulus is a figure comprising three red triangles, which is clearly not a “1-element” figure. Thus, the subject should refrain from indicating a match or correspondence.

Note that the attributes described are meant to be exemplary only, and that other attributes may be used as desired, including, for example, any of: color (e.g., red, blue, green), shape (e.g., circle, square, triangle), texture (e.g., open, solid, nested or enclosed), quantity, or the absence of any attribute, e.g., the absence of a specified color, shape, texture, or quantity, (e.g., “not blue”). Of course, other target attributes may be used as desired. Additionally, as mentioned above, in some embodiments, the target descriptor may include a plurality of target attributes, e.g., “3-elements and blue”, etc. As also mentioned above, the stimulus groups may each include multiple stimuli.

Thus, in various embodiments, the method may include one or more tasks or exercises where the target descriptor is presented, after which a continuous sequence of stimulus groups, e.g., a stream of stimuli is presented, and the subject indicates when the stimuli match or correspond to the specified target descriptor(s).

In some embodiments, the exercise may include a combination of the above tasks (possibly including other continuous performance tasks, as well). For example, in one embodiment, the presenting target descriptor (303), presenting the continuous sequence of stimulus groups (304), and the requiring, determining, and adjusting (306) may compose a session, as noted above. The repeating 314 (i.e., the repeating of 314 a plurality of times) may include two or more of: performing a plurality of sessions wherein each target descriptor is a word or object, performing a plurality of session wherein each target descriptor is a category, performing a plurality of sessions wherein each target descriptor comprises one or more attributes, performing a plurality of sessions wherein each target descriptor comprises the absence of one or more attributes, or performing a plurality of sessions wherein each target descriptor is a correspondence condition. Moreover, in various embodiments, the target descriptors and/or the stimuli may be presented in accordance with one or more modes, e.g., auditory, orthographic, pictorial, and/or in combinations of modes.

In some embodiments, the repeating of 314, and/or the performing the repeating of 314 a plurality of times, may occur a specified number of times each day, for a specified number of days. In other words, the subject may perform a plurality of sessions each day over a period of days, e.g., for 6 months, to improve cognition.

In some embodiments, certain information may be maintained and recorded over the course of the exercise. For example, in one exemplary embodiment, the following information may be recorded: the name of the subject; the age of the subject; the gender of the subject; the number of trial groups completed; all scores achieved during the exercise; the conditions in force for each trial group; time/date for each session; and time spent on each trial group, among others. Of course, this information is meant to be exemplary only, and other information may be recorded as desired.

In some embodiments, the method may also include performing one or more practice sessions, i.e., prior to performing the method elements described above. For example, in some embodiments, one or more practice sessions may be performed prior to the beginning of training to familiarize the subject with the nature and mechanisms of the exercise. In some embodiments, in each practice session, a specified number of trials (e.g., 1) for each of one or more practice conditions may be performed. In some embodiments, the subject may be able to invoke such practice sessions at will during the exercise, e.g., to re-familiarize the subject with the task at hand.

Graphical User Interface

As discussed above, in preferred embodiments, the exercises described herein are performed via a graphical user interface (GUI), examples of which are shown in FIGS. 4-9, although it should be noted that the GUI appearance and functionalities described herein are meant to be exemplary only, and are not intended to limit the GUIs to any particular form, function, or appearance.

As described above, in one embodiment, the GUI may include an introductory screen presenting task-specific instructions to the subject (and the target descriptor(s)), as shown in FIGS. 4, 6, and 8. In other embodiments, the target descriptor(s) may be presented concurrently with the stimulus (i.e., on the same screen or with sound). The subject may invoke initiation of the exercise when ready, e.g., by pressing the space bar, after which stimuli are presented, e.g., via the GUI, exemplary embodiments of which are shown in FIGS. 5, 7, and 9. As each of these exemplary GUIs shows, in preferred embodiments, the GUI may include various indicators that may operate to indicate the subject's progress and/or performance in the exercise or task. For example, turning again to FIG. 4, as may be seen, in this embodiment, the GUI includes a stimulus display area (here shown displaying the word “dollar”). The indicators shown include a score indicator, labeled “SCORE”, indicating the current points achieved in the exercise, a bonus meter or indicator, labeled “BONUS”, that may indicate how close the subject is to achieving a bonus award (e.g., bonus points), and a threshold meter or indicator, labeled “THRESHOLD”, indicating the current value of the duration and/or ISI in the exercise. Of course, in other embodiments, other indicators may be used as desired. For example, in some embodiments, the GUI may include one or more of: a total time indicator, indicating the time the subject has spent so far in the exercise, hit and miss indicators, respectively indicating the number of hits (i.e., true positives) and misses (i.e., false negatives), and a false positive indicator, indicating the number of false positives. Of course, in other embodiments, other indicators may be used as desired.

The following relates the subject's responses, described above, to aspects of the GUI, particularly the trial-by-trial rewards presented by or via the GUI, according to one embodiment, although it should be noted that these awards are meant to be exemplary only. For example, note that the responses described below are with respect to embodiments where the subject is to inhibit responses to non-targets. Embodiments where the subject is to respond respectively to targets and non-targets may use similar rewards.

Trial-By-Trial Rewards

Hit (true positive): When the subject's response is a hit or true positive, meaning that the subject has correctly indicated that the stimuli in a stimulus group correspond to or match the target descriptor, the subject may be rewarded with auditory feedback, e.g., a success sound (e.g., a “ding”), visual feedback (e.g., a graphical success indication), addition of points, and/or bonus meter advances.

Non-response (true negative): When the subject's response is a non-response or true negative, meaning that the subject has correctly refrained from indicating correspondence or matching for the stimulus group, the subject may be rewarded with bonus meter advances, and after five non-responses in a row, may be rewarded with auditory feedback, e.g., a success sound (e.g., a “ding”), visual feedback (e.g., a graphical success indication, such as a displayed “checkmark”), and addition of points.

False positive: When the subject's response is a false positive, meaning that the subject has incorrectly indicated that the stimuli in a stimulus group correspond to or match the target descriptor, the subject may be rewarded (penalized) with auditory feedback, e.g., an error sound (e.g., a “thunk”), visual feedback (e.g., a graphical indication of error or failure), bonus meter reset (where progress toward a bonus is reset to zero or decreased).

Miss (false negative): When the subject's response is a false negative, meaning that the subject has incorrectly failed to indicate that the stimuli in a stimulus group correspond to or match the target descriptor, the subject may be rewarded (penalized) with a bonus meter reset (where progress toward a bonus is reset to zero or decreased), and/or a frame color change, i.e., the GUI may modify the color of the region around the stimulus or stimulus group to indicate an error. Other rewards or penalties may be used as desired, e.g., visual feedback, e.g., an “X” under the stimulus, resetting the bonus meter, and so forth.

Threshold Determination

As indicated above, the duration and/or ISI may be adjusted using an adaptive procedure. For example, in some embodiments, the duration and/or ISI may be adjusted using a maximum likelihood procedure. Such procedures may be used to modify or set an adjustable attribute (or combination of attributes) of a presented stimulus, whereby trials in the task or exercise may be made more or less difficult. Such an adjustable parameter is generally referred to as a stimulus intensity, and the maximum likelihood procedure is used to determine a stimulus threshold, which is the value of the stimulus intensity at which the subject achieves a specified level of success, e.g., 0.9, corresponding to a 90% success rate. There are various approaches whereby such thresholds may be assessed or determined, such as, for example, the well known QUEST (Quick Estimation by Sequential Testing) threshold method, which is an adaptive psychometric procedure for use in psychophysical experiments, or a related method, referred to as the ZEST (Zippy Estimation by Sequential Testing) procedure or method, among others.

Exercise based threshold determination may be designed to assess a subject's threshold with respect to stimuli on a given exercise, and can be used to adjust stimulus presentation to achieve and maintain a desired success rate for the subject, e.g., with respect to a particular exercise, task, and/or condition. In preferred embodiments of the exercises and tasks described herein, the stimulus intensity is the duration and/or ISI of the presented stimuli. In other words, the progressions (successive modifications or adjustments of presentation parameters, e.g., duration and/or ISI) in the exercise may be calculated using an adaptive procedure, e.g., a maximum likelihood procedure, e.g., the ZEST procedure. For example, for each trial, a likelihood function may be calculated (based on the subject's response) to determine the next best guess of the true threshold. This estimate may be used (and possibly displayed) in the next trial. Based on the trial outcome and all previous trials the next best guess may be calculated and used in the presentation of stimuli for the next trial (and possibly displayed). As the probability function narrows and the standard deviation decreases, the estimate of true threshold approaches the true value—e.g., achieving a “good enough” threshold value. Note that the ZEST procedure is a modification of the QUEST procedure—the ZEST procedure uses the mean while the QUEST procedure uses the mode. In preferred embodiments, when training, a single stair ZEST procedure may be used, e.g., with a threshold level of 85%, although other values may be used as desired.

In preferred embodiments, the maximum likelihood procedure may be a continuous performance maximum likelihood procedure (as opposed to a discrete performance maximum likelihood procedure), such as a continuous performance ZEST procedure. One such a procedure, referred to as a continuous performance task (CPT), is described below.

Continuous Performance Progression

The Continuous Performance Task (CPT) is a neuropsychological task or exercise that consists of a series of stimulus groups presented one after another. The subject may be instructed to attend to a particular stimulus or a category of stimuli and respond to the presented stimulus group. The initial duration of CPT, e.g., the initial value of the presentation time for the stimulus groups, may differ from task to task.

Subject Response

In preferred embodiments, there are four possible response outcomes: true positive, true negative, false positive and false negative, defined as follows:

Correct Responses:
True positive: hit or correct response to a correct target, or
True negative: no response to a non-target (or correct response to a non-target).

Incorrect Responses:
False negative: miss or no response to a correct target, or
False positive: response to a non-target (or incorrect response to a non-target).
   Training at Optimal Level

Continuous Performance Tasks have traditionally been conducted where the stimulus Onset Asynchrony (SOA) time, e.g., the duration plus the ISI, is fixed throughout a session or is only altered after a block of stimuli is presented. Because the appropriate SOA time may differ greatly depending on: 1) the nature of the task; 2) the type of stimulus set used; and 3) the ability of the subject, a progression algorithm that optimizes the training experience is desired, especially when the training of speed of processing is crucial. For example, if the SOA time is too long, the task will not challenge the subjects and thus not engage them. If the SOA time is too short, subjects may find the task frustrating and the task may lack training value. Thus, a new SOA time progression scheme is presented herein that optimizes training experience by changing the SOA from trial to trial using an adaptive procedure, e.g., based on a maximum likelihood method. This scheme is used in preferred embodiments of the continuous performance cognitive training exercises described above.

In one embodiment, the progression scheme employed is based on the ZEST (Zippy Estimation by Sequential Testing) Bayesian adaptive estimator method, which is a probabilistic procedure where a prior estimate value of a variable is updated sequentially via a likelihood function that contains all the previous trials' information, yielding a posterior estimate value that encompasses all the data generated so far from the initial assumptions to all the user's responses. In other embodiments, other adaptive procedures may be used, e.g., QUEST.

Psychometric Function

The procedure may begin with a predetermined psychometric function, which describes the relationship between a parameter of a stimulus and the behavior of a person's response about a certain attribute of that stimulus. The psychometric function is generally a sigmoidal function, with the percentage of correct responses plotted against the stimulus parameter.

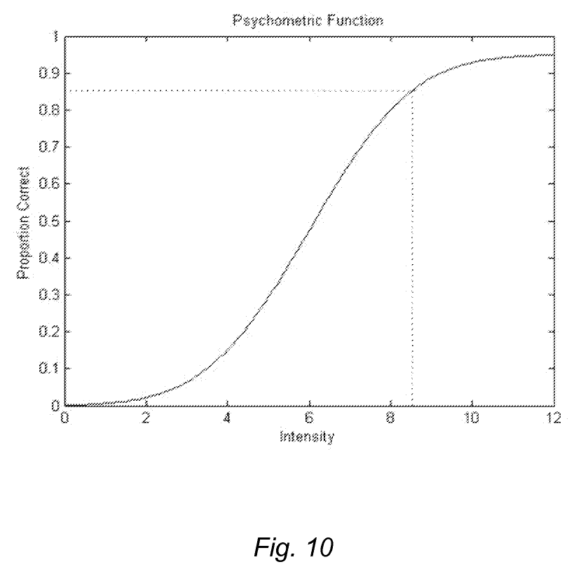

FIG. 10 illustrates an exemplary psychometric function based on a cumulative Gaussian distribution for a detection (yes/no) task with a 5% lapsing rate, and a target performance rate of 85%. In this example, the difficulty of the task decreases as the intensity parameter increases, as shown by the higher percent correct for higher parameter values. The dotted lines, at 8.5 intensity and 0.85 proportion correct, indicate the correspondence between the intensity parameter and the 85% performance rate.

The method makes several assumptions about the psychophysics:

1. The psychometric function has the same shape, except a shift along the stimulus intensity axis to indicate different performance values.

2. The performance value does not change from trial to trial.

3. Individual trials are statistically independent.

It should be noted, however, that in preferred embodiments, the methods described herein may be robust to violations of these rules, particularly to rules 2 and 3.

Prior Probability Density Function

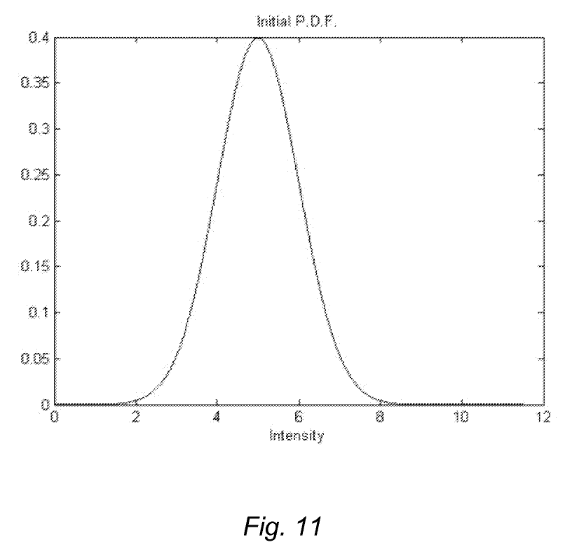

The prior probability density function (P.D.F.) is the initial distribution of the intensity values that yield the performance level in the psychophysical task. Since the function is a P.D.F., a probability function, it is a nonnegative function with the area under the curve summing up to 1, i.e., the total probability equals 100%. Typical examples of prior P.D.F.s are Gaussian distributions, Poisson distributions, Weibull distributions, and rectangular distributions, although other distributions may be used as desired. The initial estimate of intensity is often taken to be the mean of the P.D.F. FIG. 11 illustrates an exemplary probability density function with initial intensity estimate x=5, indicated by the peak of the function.

Likelihood Function

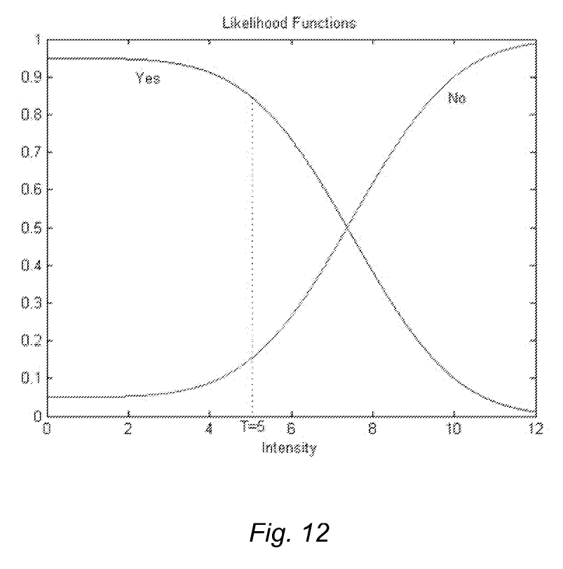

The prior P.D.F. may be adjusted after each trial by one of two likelihood functions, which are the respective probability functions describing the subject's likelihood of responding “yes” or “no” to the stimulus at the intensity as a function of the intensity. Since the psychometric function has a constant shape and the form F(x−T), fixing the intensity x and treating intensity T as the independent variable, the “yes” likelihood, p=F(−(T−x)), is thus the mirror image of the psychometric function about T, and the “no” likelihood function is then simply 1-p. FIG. 12 illustrates exemplary likelihood functions with T=5 and an 85% correct rate, correspondingly labeled “Yes” and “No”.

Posterior Probability Density Function

After a presentation is complete and the response is noted, the prior P.D.F. may be updated using Bayes' rule, by multiplying the prior P.D.F. by the likelihood function corresponding to the subject's response to the trial's stimulus intensity to obtain the posterior P.D.F. The mean of the posterior P.D.F. may then be used as the new intensity estimate. This procedure may be repeated after every presentation to determine the subsequent intensity of the stimulus.

Terminating Condition

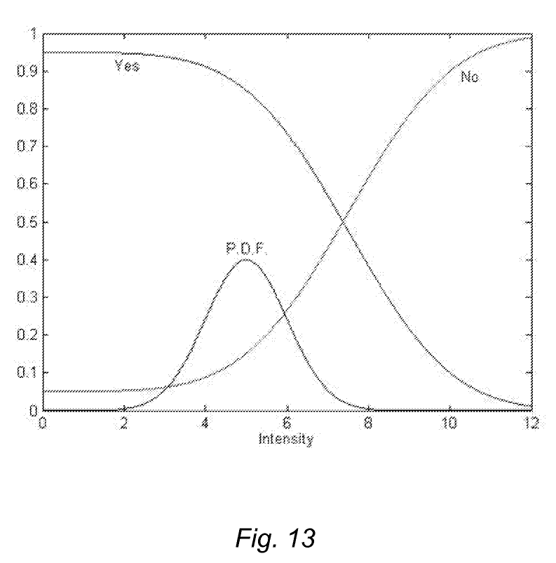

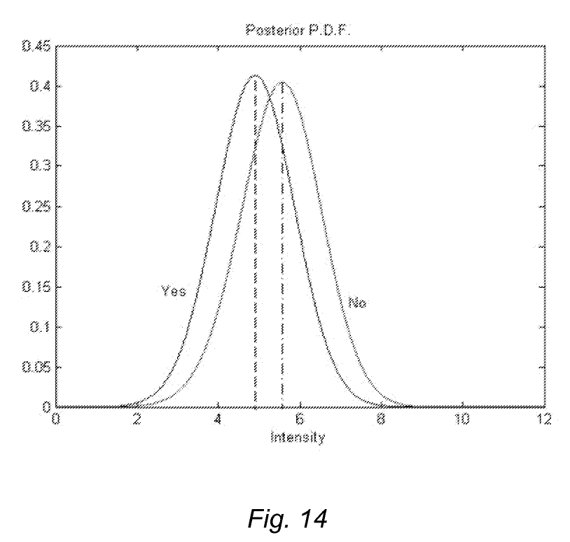

The procedure may be terminated if the posterior P.D.F. satisfies a certain confidence level. This may first require the posterior P.D.F. computed to be normalized. If the confidence interval of the posterior P.D.F. yielding the confidence level is less than the specified length, then the procedure may be terminated, and the final intensity obtained is the threshold intensity. FIG. 13 illustrates an exemplary prior P.D.F. (labeled “P.D.F.”) superimposed with exemplary likelihood functions (again, labeled “Yes” and “No”). FIG. 14 illustrates an exemplary posterior P.D.F. with Yes/No response. Note that the intensity for the respective Yes and No P.D.F. functions differs.

CPT Framework

In one embodiment of the continuous performance framework described herein, illustrated by FIGS. 10-14, an SOA time (represented by intensity) is sought that results in the subject achieving an 85% correct rate, with the initial SOA time set at 5 units. In this example, if the response to the trial is correct, the next SOA time is updated to ˜4.8, while an incorrect response yields subsequent SOA time ˜5.5. This new SOA time may then be used for the presentation duration of the next stimulus (e.g., stimulus group) in the block or sequence (stream). The procedure may continue in the same fashion for the updated SOA time to yield the subsequent SOA time for the next presentation. Note, however, that these particular values are meant to be exemplary only, and that any other values may be used as desired.

Thus, various embodiments of the methods disclosed herein may utilize an adaptive procedure, e.g., a continuous performance maximum likelihood procedure, e.g., continuous performance ZEST, to adaptively modify one or more stimulus presentation parameters, e.g., duration and/or ISI, in continuous performance tasks or exercises utilizing stimulus streams.

It should also be noted that the particular exercises and tasks disclosed herein are meant to be exemplary, and that other continuous performance cognitive training exercises using stimulus streams may be used as desired, possibly in combination. In other words, the exercises and tasks described herein are but specific examples of cognitive training exercises and tasks using a computing system to present one or more stimulus streams to a subject, record the subject's responses, and modify some aspect of the stimuli based on these responses, where these method elements are repeated in an iterative manner using multiple sets of stimuli to improve the subject's cognition. Note particularly that such cognitive training using a variety of such stimulus stream-based exercises or tasks, possibly in a coordinated manner, is contemplated. Thus, various embodiments of the cognitive training exercises and tasks described herein may be used singly or in combination to improve the subject's cognitive skills.

Those skilled in the art should appreciate that they can readily use the disclosed conception and specific embodiments as a basis for designing or modifying other structures for carrying out the same purposes of the present invention without departing from the spirit and scope of the invention as defined by the appended claims. For example, various embodiments of the methods disclosed herein may be implemented by program instructions stored on a memory medium, or a plurality of memory media.

We claim:

1. A computer-implemented method for enhancing cognition in a subject, utilizing a computing device to present stimuli and to receive responses from the subject, the method comprising:
a) providing a set of stimuli for presentation to the subject;
b) presenting a target descriptor to the subject, wherein the target descriptor specifies attributes of a plurality of stimuli from the set of stimuli;
c) presenting a continuous sequence of stimulus groups from the set of stimuli to the subject one stimulus group at a time, wherein each stimulus group comprises at least one stimulus, wherein each stimulus group is presented for a specified duration, and wherein the stimulus groups in the continuous sequence of stimulus groups are separated by a specified inter-stimulus-interval (ISI);
d) for each stimulus group in the continuous sequence of stimulus groups,
requiring the subject to respond to the stimulus group by indicating when the at least one stimulus in the stimulus group corresponds to the target descriptor;
determining if the subject responded correctly for the stimulus group;
adjusting the duration and/or the ISI based on said determining, wherein said adjusting the duration and/or the ISI is performed using an adaptive procedure; and
e) repeating b)-d) one or more times in an iterative manner to improve the cognition of the subject.

2. The method of claim 1, wherein b)-d) is performed under a specified condition, wherein the condition specifies one or more attributes of said presenting the continuous sequence of stimulus groups, the method further comprising:
repeating b)-e) a plurality of times in an iterative manner, wherein, for each iteration, b)-e) is performed under a respective condition.

3. The method of claim 2, wherein each condition specifies one or more of:
length of the continuous sequence of stimulus groups;
length of time of said presenting the continuous sequence of stimulus groups;
correspondence frequency, comprising a ratio of stimulus groups in which the at least one stimulus in the stimulus group corresponds to the target descriptor to stimulus groups in which the at least one stimulus in the stimulus group does not correspond to the target descriptor; or
target/foil confusability, comprising a degree to which stimuli that correspond to the target descriptor are similar to stimuli that do not correspond to the target descriptor.

4. The method of claim 1, further comprising:
indicating whether the subject responded correctly, wherein said indicating is performed audibly and/or visually.

5. The method of claim 4, further comprising:
requiring the subject to respond to the stimulus group by refraining from indicating when the at least one stimulus in the stimulus group does not correspond to the target descriptor.

6. The method of claim 5, wherein the subject's response to the stimulus group comprises one of:
a correct response, comprising:
a true positive, wherein the subject correctly indicates when the at least one stimulus in the stimulus group corresponds to the target descriptor; or
a true negative, wherein the subject correctly refrains from indicating when the at least one stimulus in the stimulus group does not correspond to the target descriptor; or
an incorrect response, comprising:
a false negative, wherein the subject fails to indicate when the at least one stimulus in the stimulus group corresponds to the target descriptor; or
a false positive, wherein the subject incorrectly indicates that the at least one stimulus in the stimulus group corresponds to the target descriptor.

7. The method of claim 4, further comprising:
requiring the subject to respond to the stimulus group by indicating when the at least one stimulus in the stimulus group does not correspond to the target descriptor.

8. The method of claim 7, wherein the subject's response to the stimulus group comprises one of:
a correct response, comprising:
a true positive, wherein the subject correctly indicates when the at least one stimulus in the stimulus group corresponds to the target descriptor; or
a true negative, wherein the subject correctly indicates when the at least one stimulus in the stimulus group does not correspond to the target descriptor; or
an incorrect response, comprising:
a false negative, wherein the subject incorrectly indicates that the at least one stimulus in the stimulus group does not correspond to the target descriptor; or
a false positive, wherein the subject incorrectly indicates that the at least one stimulus in the stimulus group corresponds to the target descriptor.

9. The method of claim 6, wherein said requiring the subject to respond comprises requiring the subject to respond before a next stimulus group is presented.

10. The method of claim 4, wherein said indicating whether the subject responded correctly comprises:
rewarding the subject if a specified level of success is achieved; or
penalizing the subject if a specified level of failure is achieved;
wherein the rewarding and penalizing each comprises one or more of:
auditory feedback;
visual feedback;
point modification; or
change in bonus status.

11. The method of claim 4, further comprising:
terminating said repeating b)-d) if the subject responds incorrectly a specified number of times consecutively.

12. The method of claim 1, wherein b)-d) composes a session, the method further comprising:
presenting a reward to the subject at the end of each session, wherein the reward is presented graphically and/or audibly.

13. The method of claim 1, wherein the adaptive procedure comprises a maximum likelihood procedure.

14. The method of claim 13, wherein the maximum likelihood procedure comprises a continuous performance maximum likelihood procedure.

15. The method as recited in claim 14, wherein the continuous performance maximum likelihood procedure comprises one or more of:
a continuous performance QUEST (quick estimation by sequential testing) threshold procedure; or
a continuous performance ZEST (zippy estimation by sequential testing) threshold procedure.

16. The method of claim 1, wherein said adjusting the duration and/or the ISI comprises:
adjusting the duration and/or the ISI to approach and substantially maintain a specified success rate for the subject.

17. The method of claim 16, wherein said adjusting the duration and/or the ISI to approach and substantially maintain a specified success rate for the subject uses a single-stair continuous performance maximum likelihood procedure.

18. The method of claim 1, wherein b)-d) compose a session, and wherein for each session, an initial value of the duration and/or the ISI and a final value of the duration and/or the ISI are averaged to determine the initial value of the duration and/or the ISI for the next session.

19. The method of claim 1, wherein b)-d) compose a session, and wherein for each session, reaction times for each stimulus group in the continuous sequence of stimulus groups are averaged to determine a minimum value for the duration and/or the ISI for the next session, wherein each reaction time comprises a respective delay between the presentation of each stimulus group corresponding to the target descriptor and the subject's response to the stimulus.

20. The method of claim 1, wherein each stimulus group presentation and corresponding subject response composes a trial, the method further comprising:
for each trial, recording of one or more of:
the target descriptor;
the stimulus group;
whether or not the stimulus group corresponds to the target descriptor;
the duration;
the ISI;
the subject's response;
the correctness or incorrectness of the subject's response;
a reaction time for the trial, comprising the delay between the presentation of a stimulus group and the subject's response; or
statistical measures for the adaptive procedure.

21. The method of claim 1, wherein b)-d) compose a session, the method further comprising:
performing an initial session prior to performing b)-d), wherein the initial session comprises b)-d), but wherein, in performing d) in the initial session, neither the duration nor the ISI is adjusted.

22. The method of claim 1, wherein the target descriptor is presented in a specified mode, comprising one or more of:
an auditory mode, wherein the target descriptor is presented audibly;
a pictorial mode, wherein the target descriptor is presented pictorially; or
an orthographic mode, wherein the target descriptor is presented textually.

23. The method of claim 1, wherein the continuous sequence of stimulus groups is presented in a specified mode, comprising one or more of:
an auditory mode, wherein each stimulus group is presented audibly;
a pictorial mode, wherein each stimulus group is presented pictorially; or
an orthographic mode, wherein each stimulus group is presented textually.

24. The method of claim 1, wherein the target descriptor comprises one or more of:
a word or object;
a category;
one or more attributes;
absence of one or more attributes; or
a correspondence condition.

25. The method of claim 24, wherein each of at least a subset of the stimuli in the set of stimuli comprises a plurality of attributes, comprising two or more of:
color;
shape;
texture;
quantity;
absence of a specified color;
absence of a specified shape;
absence of a specified texture; or
absence of a specified quantity.

26. The method of claim 24, wherein b)-d) composes a session, the method further comprising:
repeating b)-e) a plurality of times in an iterative manner, wherein said repeating comprises two or more of:
performing a plurality of sessions wherein each target descriptor is a word or object;
performing a plurality of sessions wherein each target descriptor is a category;
performing a plurality of sessions wherein each target descriptor comprises one or more attributes;
performing a plurality of sessions wherein each target descriptor comprises the absence of one or more attributes; or
performing a plurality of sessions wherein each target descriptor is a correspondence condition.

27. The method of claim 1, wherein the at least one stimulus in the stimulus group comprises a plurality of stimuli, wherein the target descriptor comprises one or more relationships of attributes of the plurality of stimuli, each relationship comprising one or more of:
identical, wherein each stimulus of the plurality of stimuli has a common attribute value; or
distinct, wherein each stimulus of the plurality of stimuli has a different attribute value;
wherein the attributes of the plurality of stimuli comprises two or more of:
color;
shape;
texture;
quantity;
absence of a specified color;
absence of a specified shape;
absence of a specified texture; or
absence of a specified quantity.

28. The method of claim 1, further comprising:
repeating b)-e) a plurality of times in an iterative manner, wherein said repeating b)-e) occurs a specified number of times each day, for a specified number of days.

29. The method of claim 1, wherein b)-d) compose a session, the method further comprising:
performing an initial session prior to performing b)-d), wherein the initial session comprises b)-d), but wherein, in performing d) in the initial session, neither the duration nor the ISI is adjusted.

30. A computer accessible memory medium comprising program instructions for enhancing cognition in a subject, utilizing a computing device to present stimuli and to receive responses from the subject, wherein the program instructions are executable by a processor to perform:
a) providing a set of stimuli for presentation to the subject;
b) presenting a target descriptor to the subject, wherein the target descriptor specifies attributes of a plurality of stimuli from the set of stimuli;
c) presenting a continuous sequence of stimulus groups from the set of stimuli to the subject one stimulus group at a time, wherein each stimulus group comprises at least one stimulus, wherein each stimulus group is presented for a specified duration, and wherein the stimulus groups in the continuous sequence of stimulus groups are separated by a specified inter-stimulus-interval (ISI);
d) for each stimulus group in the continuous sequence of stimulus groups,
requiring the subject to respond to the stimulus group by indicating when the at least one stimulus in the stimulus group corresponds to the target descriptor;
determining if the subject responded correctly for the stimulus group;
adjusting the duration and/or the ISI based on said determining, wherein said adjusting the duration and/or the ISI is performed using an adaptive procedure; and
e) repeating b)-d) one or more times in an iterative manner to improve the cognition of the subject.

31. The method of claim 30, wherein b)-d) is performed under a specified condition, wherein the condition specifies one or more attributes of said presenting the continuous sequence of stimulus groups, the method further comprising:
repeating b)-e) a plurality of times in an iterative manner, wherein, for each iteration, b)-e) is performed under a respective condition.
